(19) United States
(12) Patent Application Publication
(10) Pub. No.: US 2007/0243539 A1
(43) Pub. Date: Oct. 18, 2007

(54) GENETIC VARIANTS OF HUMAN INOSITOL POLYPHOSPHATE-4-PHOSPHATASE, TYPE I (INPP4A) USEFUL FOR PREDICTION AND THERAPY OF IMMUNOLOGICAL DISORDER

(76) Inventors: Balaram Ghosh, Delhi (IN); Mamta Sharma, Delhi (IN); Jyotsna Batra, Delhi (IN)

Correspondence Address: MORGAN & FINNEGAN, L.L.P., 3 World Financial Center, New York, NY, 10281-2101, US

(21) Appl. No.: 11/552,769

(22) Filed: Oct. 25, 2006

(30) Foreign Application Priority Data
Oct. 25, 2005 (IN) 1536/DEL/2005

(51) Int. Cl. C12Q 1/68; C07H 21/02; G01N 33/00; C07H 21/04

(52) U.S. Cl. 435/6; 436/94; 536/23.1

(57) ABSTRACT

Atopic asthma is a chronic, inflammatory lung disease characterized by recurrent breathing problems in response to an allergen. Platelets play an important role in this allergic inflammatory process, by releasing preformed mediators like platelet factor 4 (PF4) and regulated upon activation in normal T cells expressed and secreted (RANTES) upon activation causing eosinophil chemotaxis. The present invention relates to allelic variants of the human Inositol polyphosphate 4-phosphatase (INPP4A) gene and splice variants of the coding sequence, which encodes INPP4A enzyme known to be an important regulator of platelet activation; and provides primers and methods suitable for the detection of these allelic variants for applications such as molecular diagnosis, prediction and prevention of an individual's disease susceptibility, and/or the genetic analysis of the INPP4A gene in a population. The invention also provides an association with the expression profile of INPP4A protein in the mouse model of asthma. Specifically, the invention provides a method for detection of predisposition to atopic disorders/other immunological disorders such as, autoimmune disorders, inflammatory disorders, cancer, multiple sclerosis, fibrosis, tuberculosis, sarcoidosis, hypertension and disorders developing due to hypertension, diabetes and disorders developing due to diabetes, alcohol abuse, anxiety, asthma, chronic obstructive pulmonary disease (COPD), cholecystectomy, degenerative joint disease (DJD), seizure disorder, arthritis, etc. where human Inositol polyphosphate 4-phosphatase (INPP4A) might play an important role due to its involvement in platelet action.

### FIELD OF THE INVENTION

The present invention relates to the genetic variants of human Inositol polyphosphate 4-phosphatase (INPP4A) gene useful for the prediction and therapy of immunological disorders. More particularly, the present invention relates to genetic and splice variants of the coding sequence of the human Inositol polyphosphate 4-phosphatase (INPP4A) gene. The invention further provides primers and methods suitable for the detection of these allelic variants for the prediction of an individual's susceptibility to diseases and/or the genetic analysis of the INPP4A gene for immunological disorders, particularly asthma.

### BACKGROUND AND PRIOR ART REFERENCES OF THE PRESENT INVENTION

Asthma is a common chronic airway disease, with considerable heterogeneity both in its phenotype and in the underlying pathophysiology. It affects 15-18% of the world's population. Both intrinsic and extrinsic cases of asthma are known. Intrinsic asthma is mainly childhood disorder though the age of onset can vary and is seen to be 35-45 years in the general population; whereas extrinsic asthma is observed where the age of onset is above 45 yr. and is mainly due to the age induced changes in the lung function.

Recent studies have demonstrated that airway inflammation is a principal feature in the pathophysiology of asthma (Gern et al. 1999). The disorder is multifactorial (in both initiation and progression) because of the involvement of numerous resident and recruited inflammatory cells. T cells and IgE-mediated responses are known to be a key factor in the allergic response (Elias et al. 2003). Asthma is a T helper type 2 (Th2) mediated disorder with cytokines such as interleukin-4, interleukin-5, interleukin-13, implicated in the deviation of the immune system towards atopicity. Increased levels of these cytokines lead to elevated total serum IgE levels, eosinophil recruitment, and bronchial hyper-responsiveness that ultimately culminate in asthma pathogenesis. These interleukins are also known to interact and stimulate the alveolar cells and bronchial smooth muscle cells resulting in the clinical phenotypes of bronchial hyper-responsiveness (Barnes P J, Respir Res 2:64-5, 1999). Gene-gene and gene-environment interactions have been implicated in the development of asthma (Tay et al, Asian Pac J Allergy Immunol 17:239-42, 1999; Bleecker E R, Am J Respir Crit Care Med 156:S113-6, 1997; Cookson W, Nature 402:B5-11, 1999).

Inflammation and airway remodeling are characteristic features of atopic asthma. Various types of cells viz: eosinophils, mast cells, and T lymphocytes migrate to the lungs and mediate the inflammation process at the site (Barnes P J, 1992). In addition to these cells, platelets play a key role in this allergic inflammatory process, because they are a rich source of a wide range of biologically active materials capable of inducing or augmenting allergic inflammatory responses (Herd C M, 1994, Klinger, 1995). Such materials have been demonstrated to be preformed mediators stored in a-granules, which are chemokines such as platelet factor 4 (PF4) and regulated upon activation in normal T cells expressed and presumably secreted (RANTES) (Herd C M, 1994, Klinger, 1995). These chemokines are released from platelets after stimulation with potent anaphylactic mediators such as platelet activating factor (PAF) (Herd C M, 1994, Kameyoshi Y, 1992) and cause eosinophilic chemotaxis (Kameyoshi Y, 1992), providing additional evidence for a contribution of platelets to bronchial asthma. Studies in mouse model of allergic inflammation suggest the role of platelets in airway remodeling (Simon C et al, 1994). Platelet activation has been found to be associated with inactivation of a magnesium independent enzyme Inositol polyphosphate 4-phosphatase (INPP4A, EC 3.1.3.66) (Norris F A, 1997). Stimulation of human platelets with thrombin or calcium ionophore results in inactivation of INPP4A by proteolytic cleavage by calcium dependent protease calpain (Norris F A et al, 1997). The enzyme INPP4A catalyzes the hydrolysis of the 4-position phosphate of inositol 3,4-bisphosphate and inositol 1,3,4-trisphosphate. It also catalyzes, at a much higher rate, the hydrolysis of the 4-position phosphate of phosphatidylinositol 3,4-bisphosphate generated by the phosphorylation at the D-3 position of inositol lipids by Phosphoinositide 3-kinases (PI3Ks). Thus inactivation of INPP4A is associated with calcium/aggregation dependent accumulation of PtdIns (3,4) P2 characteristic of stimulated human platelets. Norris et al. (1995) noted that INPP4A is also implicated in mitogenesis mediated by PDGF receptor, the oxidative burst of neutrophils, translocation of the glucose transporter to the plasma membrane. Vyas P et al 2000 found that INPP4A regulates cell proliferation downstream of GATA-1 transcription factor. GATA-1⁻ megakaryocytes (precursor of platelets) were found to be deficient in this enzyme and showed hyperproliferation. Reintroduction of INPP4A into GATA-1⁻ megakaryocytes significantly retarded cell growth, suggesting the role of this enzyme in cell proliferation. When differential gene expression was compared between lungs of ovalbumin sensitized/challenged A/J mice versus control mice, various genes were found to be differentially expressed (Gene Expression Omnibus Database (GEO), GDS 349). Among these differentially expressed genes, INPP4A was also found to be modulated in the lungs of sensitized mice.

The INPP4A gene encodes two protein isoforms α and β varying in their carboxy terminus and various splice variants are also known in the exon 17, 18 and 19 region of this gene (NT022171), earlier designated as exon 15, 16 and 17 (Shearn C T et al, 2001). Two more exons encoding 5′ UTR have been annotated in the contig NT022171. There are 3 splice forms viz; α1, 2 and 3 reported in the exon 17-19 region as shown in FIG. 1A.

INPP4 α1 encodes a 106 kDa form of the major enzyme expressed in human, rat and mouse brain (Norris F A et al, 1995; Vyas P et al, 2000). INPP4 α2 encodes a 102 kDa form that is minor species expressed in rat and mouse brain (Norris F A et al, 1995). The α3 isoform results from the use of an alternative 5′-GU splice donor site during the excision of intron 17 and extends the exon 17 by 120 bp and encodes a 110 kDa protein expressed as the major form in human platelets (Shearn C T et al, 2001). This extended exon 17 region contains three repeats spaced seven bases apart with the nucleotide sequence CCCCTYCW where Y represents C or T and W represents A or T. Exon 18 also contains a sequence with this consensus. These consensus pyrimidine rich elements represent recognition sites for splicing factors that regulate the tissue specific alternative splicing of exons 17 and 18. The extended exon 17 encodes a 40 amino acid domain, which contains PEST sequence. PEST sequences are rich in proline, serine, glutamate/aspartate and threonine residues and the proteins containing such sequences are rapidly degraded by the calpain family of proteases (Rogers S et al, 1986; Rechsteiner M and Rogers S 1996).

INPP4A enzyme containing PEST sequences also is rapidly degraded by calpain family of proteases, which act on proteins containing PEST sequences (Norris F A et al, 1997). As the stimulation of platelets is associated with asthma, which in turn correlated with inactivation of the INPP4A enzyme, we hypothesized that gene variants of INPP4A could be associated with immunological disorders including asthma. However, no studies have been done till date to study the genetic role of INPP4A in immunological disorders including atopic asthma.

### NOVELTY OF THE INVENTION

The inventors for the first time have provided INPP4A gene variants associated with the immunological disorders including asthma in humans.

The novelty of the invention is in the provision of disease association of INPP4A +92031 A/T (S1), +92344 C/T (S2), +92817 C/T (S3), +110832 A/G (S4), +131237 C/T (S5) single nucleotide polymorphisms and D2S2311 (M1), D2S2187 (M2) and +99095 CA (M3) repeat in the intronic and exonic and flanking regions of the gene.

Another novelty is in the identification of the risk associated with the genetic variants for asthma, which are preferentially transmitted to the affected offspring.

Still another novelty is in the prediction of susceptibility to immunological disorders including asthma associated with +110832 A/G (Ala/Thr) SNP in the α3-splice variant and its role in regulation of calapain proteases.

Here the applicants have for the first time provided gene variants and novel haplotypes of INPP4A gene useful for prediction of immunological disorders, particularly asthma. They have also shown the importance of the expression of INNP4A protein for alleviating asthmatic condition in the mouse model of asthma.

### OBJECTS OF THE INVENTION

The main object of the present invention is to provide genetic variants of human Inositol polyphosphate 4-phosphatase (INPP4A) gene useful for prediction and therapy of immunological disorders including atopic asthma.

Another object of the present invention is to provide a method for predicting an individual's susceptibility for immunological disorders particularly asthma by screening for INPP4A gene variants.

Still another object of the invention is to provide a method for screening disease prone individuals in a given population by screening for the INPP4A gene variants.

Yet another object is to provide specific primers for detection of single nucleotide polymorphisms and specific repetitive sequences in and around the INPP4A gene.

Another object is to define the haplotypes generated by the allelic variants of the INPP4A gene in the general (control) population.

Still another object of the invention is to provide a method for studying association of the haplotypes of the INPP4A allelic variants with disease susceptibility.

Another object of the invention is to identify splice variants of the INPP4A gene expressed in asthmatic individuals.

Yet another object of the invention is to provide a method for predicting an individual's risk towards developing immunological disorders, specifically atopic asthma, by studying the haplotype pattern.

Still another object of the invention is to find the role of INPP4A gene variant in protein stability.

Another object of the invention is to establish the expression differences of INPP4A gene in mouse model of asthma as compared to the saline treated control mice.

Still another object is to the restoration of the INPP4A normal levels in mouse model of asthma after treatment with anti-inflammatory drugs such as steroid.

Yet another object of the invention is to provide a INPP4A protein profile based method for prediction of susceptibility to atopic asthma.

### SUMMARY OF THE INVENTION

Atopic asthma is a chronic, inflammatory lung disease characterized by recurrent breathing problems in response to an allergen. Platelets play an important role in this allergic inflammatory process, by releasing preformed mediators like platelet factor 4 (PF4) and regulated upon activation in normal T cells expressed and secreted (RANTES) upon activation causing eosinophil chemotaxis. The present invention relates to allelic variants of the human Inositol polyphosphate 4-phosphatase (INPP4A) gene and splice variants of the coding sequence, which encodes INPP4A enzyme known to be an important regulator of platelet activation; and provides primers and methods suitable for the detection of these allelic variants for applications such as molecular diagnosis, prediction and therapy of an individual's disease susceptibility, and/or the genetic analysis of the INPP4A gene in a population. The invention also provides an association with the expression profile of INPP4A protein in the mouse model of asthma.

The invention also provides a method for detection of predisposition to atopic disorders/other immunological disorders such as, autoimmune disorders, inflammatory disorders, cancer, multiple sclerosis, fibrosis, tuberculosis, sarcoidosis, hypertension and disorders developing due to hypertension, diabetes and disorders developing due to diabetes, alcohol abuse, anxiety, asthma, chronic obstructive pulmonary disease (COPD), cholecystectomy, degenerative joint disease (DJD), seizure disorder, arthritis, etc. where human Inositol polyphosphate 4-phosphatase (INPP4A) might play an important role.

### DETAILED DESCRIPTION OF THE INVENTION

In the present invention INPP4A has been identified as a candidate gene for finding association with immunological disorders, particularly asthma as several polymorphisms in this gene were found to be associated with atopic asthma in case-control and family studies.

The INPP4A gene encodes a magnesium independent enzyme Inositol polyphosphate 4-phosphatase, which catalyzes the hydrolysis of the 4-position phosphate of inositol 3,4-bisphosphate and inositol 1,3,4-trisphosphate. It also catalyzes, at a much higher rate, the hydrolysis of the 4-position phosphate of phosphatidylinositol 3,4-bisphosphate generated by the phosphorylation at the D-3 position of inositol lipids by Phosphoinositide 3-kinases (PI3Ks). The INPP4A gene is expressed in humans in various tissues viz. Bladder, Blood, Bone, Bone Marrow, Brain, Cervix, Colon, Eye, Heart, Kidney, Liver, Lung, Lymph Node, Mammary Gland, Muscle, Ovary, Pancreas, Placenta, Prostate, Skin, Soft Tissue, Spleen, Stomach, Testis, Thymus, Uterus (Expression profile from UniGene Cluster Hs.469386). Norris et al. (1995) noted that the latter activity of INPP4A is implicated in mitogenesis mediated by PDGF receptor, the oxidative burst of neutrophils, translocation of the glucose transporter to the plasma membrane and platelet aggregation. Stimulation of human platelets with thrombin or calcium ionophore results in inactivation of INPP4A by proteolytic cleavage by calcium dependent protease calpain (Norris F A et al, 1997). This inactivation of INPP4A is associated with calcium/aggregation dependent accumulation of PtdIns(3,4)P2 characteristic of stimulated human platelets. Thus, INPP4A is an important enzyme involved in regulation of this lipid second messenger in platelets. The platelets are known to be important players in asthma pathogenesis; many studies in the nineties have shown the importance of these cells in asthma. Platelets were found to be necessary for airway remodeling in mouse model of asthma (Simon C et al, 2004) and promote eosinophil adhesion to endothelium in asthmatic individuals (Ulfman L H et al, 2003).

The present application deals with D2S2311; a microsatellite marker, 44.7 kb upstream of the gene, dinucleotide polymorphic repeat at nucleotide 99095 to 99127, which is in the intron 11 of the INPP4A gene (GenBank accession no. NT_{—}022171). The first three SNPs (rs3769712, rs3769710, rs2278208), as shown in FIG. 1B, are 92031 bp, 92344 bp and 92817 bp downstream of the gene start site respectively and lie in intron 7 of the gene. The fourth non-synonymous SNP (rs2278206) is situated 110832 bp downstream of the human INPP4A gene in alternatively spliced exon 17 and represents a A to G transition (Thr to Ala). The fifth SNP rs10201079 is a C to T transition, situated 131237 bases downstream of the INPP4A gene start site in the intron 24.

The results of the present study provide very unique results. In addition to identifying, genotyping and establishing a positive association of the microsatellite D2S2311, 44.7 kb upstream of the gene with asthma, the inventors have found an association of the known +92031 A/T, +110832 A/G, +131237 C/T single nucleotide polymorphisms and a dinucleotide repeat at +99095 in the Indian population. All the SNPs and microsatellite repeats have been validated in Indian population for the first time.

The present invention has identified the genetic variants, which exist in any type of population in the world irrespective of its origin, community, colour, geographical location or ethnicity. The inventors have compared allele and genotype frequencies of D2S2311 repeat polymorphism 44.7 Kb upstream of promoter, D2S2187 in intron 1 at +43987 position +92031 A/T, +92344 C/T, +92817 C/T SNPs in intron 7, CA repeat at +99095 in the intron 11, +110832 A/G in splice variant exon 17 and +131237 C/T in intron 24 and the haplotypes generated using four loci, in Indian population. In the present study both case control and family based association study was carried out for these polymorphisms. Further, the invention clearly defines that the variants identified would be useful for any kind of population of any geographical origin.

The present invention has also identified the functional role of +110832 A/G polymorphism in splice variant exon 17 expressed in platelets. The extended exon contains potential PEST sequence from amino acid 584-607 with PESTfind score of +7.49 (www.at.embnt.org/embnet/tools/bio/pestfind/). The +110832 A/G (rs2278206) polymorphism causes a threonine to alanine substitution at position 604 in protein sequence resulting in a poor PEST sequence (PESTfind score +4.95). Thus a A to G base substitution at this particular locus can make the INPP4 enzyme resistant to calpain proteases as shown by western blot experiments from human platelets.

The present invention also reports the identification of novel splice variants in asthmatic individuals. One of the splice variants has deleted exon 16 of 219 base pairs. This results in deletion of 73 amino acids from the encoded protein. This splice variant can result in protein with altered function.

The present invention also shows the lower expression of INPP4A protein in the allergen—induced mouse model of asthma than in the saline treated normal mice and its restoration after treatment with anti-inflammatory drugs such as steroid.

Accordingly, the present invention provides genetic variants of human Inositol polyphosphate 4-phosphatase (INPP4A) gene useful for prediction and therapy of immunological disorders, particularly asthma, said variants:
(a) The gene variant of SEQ ID No. 1 has 1-230 contiguous nucleotides containing group of CA dinucleotides of locus M1 present 44.7 kb upstream of gene start site.
(b) The gene variant of SEQ ID No. 2 has 1-400 contiguous nucleotides containing GT dinucleotides at locus M2.
(c) The gene variant of SEQ ID No. 3 has 1-159 contiguous nucleotides containing CA repeat polymorphism at nucleotide 229 of M3 locus.
(d) The gene variant of SEQ ID No. 4 has 1-1036 contiguous nucleotides containing A/T polymorphism at nucleotide 75 of locus S1, C/T polymorphism at nucleotide 388 of locus S2 and C/T polymorphism at nucleotide 861 of locus S3.
(e) The gene variant of SEQ ID No. 5 has 1-961 contiguous nucleotides containing G/A polymorphism at nucleotide 147 of S4 locus.
(f) The gene variant of SEQ ID No. 6 has 1-1707 contiguous nucleotides containing C/T polymorphism at nucleotide 1221 of S5 locus.
(g) The splice variant of SEQ ID No. 24 has 1-817 contiguous nucleotides containing splice variant region.

Another aspect of the invention relates to a method of detecting gene variants having SEQ ID Nos. 1, 2, 3, 4, 5, 6 and 24 of INPP4A gene and expressed cDNA splice variant having SEQ ID 24 for detecting and predicting susceptibility of a subject to immunological disorders. The said method comprising the steps of:
1) Isolating the genomic DNA from peripheral blood leucocytes (PBL) and PCR amplifying the 230 bp DNA stretch of SEQ ID No.1, 44.7 kb upstream of INPP4A gene using novel primers of SEQ ID No. 7and 8,
2) PCR amplifying the 401 bp DNA stretch of SEQ ID No. 2 of INPP4A gene intron, using novel primers of SEQ ID No. 9 and 10
3) PCR amplifying the 159 bp DNA stretch of SEQ ID No. 3 of INPP4A intron 11, using novel primers of SEQ ID No. 11 and 12
4) PCR amplifying the 1036 bp DNA stretch of SEQ ID No. 4 of INPP4A gene, using novel primers of SEQ ID No. 13 and 14
5) PCR amplifying the 961 bp DNA stretch of SEQ ID No. 5 of INPP4A gene, using novel primers of SEQ ID No. 15 and 16
6) PCR amplifying the 1707 bp DNA stretch of SEQ ID No. 6 of INPP4A gene, using novel primers of SEQ ID No. 17 and 18
7) Direct sequencing of the purified PCR products and locating sequence variants in the Seq ID 4, 5 and 6 for +92031 A/T, +92344 C/T, +92817 C/T, +110832 A/G, +131237 C/T polymorphisms by aligning the amplified DNA sequences with the already existing sequence of human INPP4A gene in the NCBI database (GenBank accession no. NT_{—}022171).
8) Genotyping the +92031 A/T, +92344 C/T, +92817 C/T, +110832 A/G, +131237 C/T polymorphisms using SnapShot primers of SEQ ID Nos. 19, 20, 21, 22, 23 respectively.
9) Identifying splice variants of INPP4A gene by Reverse transcriptase PCR using novel primers of SEQ ID No. 25 and 26.
10) Developing the mouse model of asthma by ovalbumin sensitization and challenge, and profiling INPP4A protein expression by immunohistochemistry using commercially available INPP4A antibody.

Other and further aspects, features, and advantages of the present invention will be apparent from the following description of the presently preferred embodiments of the invention given for the purpose of disclosure.

In an embodiment to the invention the gene variants of INPP4A gene comprise:

SEQ ID No.1 associated with D2S2311 locus, SEQ ID No. 2 associated with D2S2187 locus, SEQ ID No. 3 associated with +99095 CA repeat, SEQ ID No. 4 associated with +92031 A/T, +92344 C/T, +92817 C/T locus, SEQ ID No. 5 associated with +110832 A/G, SEQ ID No. 6 associated with +131237 C/T and SEQ ID No. 23 is associated with splice variants.

In another embodiment to the invention the immunological disorders selected are from group comprising of asthma, autoimmune disorders, inflammatory disorders, cancer, multiple sclerosis, fibrosis, tuberculosis, sarcoidosis, hypertension and related disorders, diabetes and related disorders, alcohol abuse, anxiety, COPD, cholecystectomy, degenerative joint disease, seizure disorders, arthritis etc.

In still another embodiment the immunological disorder is asthma.

In yet another embodiment the subject is human.

In another embodiment the variants are pharmacogenetic markers for predicting and detecting humans susceptible to immunological disorders.

In another embodiment the variants are pharmacogenetic markers for predicting and detecting humans susceptible to asthma.

Another embodiment to the present invention, the D2S2311 microsatellite allelic variants at M1 locus is associated with susceptibility to asthma χ²=43.441128, DF=9, p value<0.0001 and its association is confirmed in family based studies (p=0.0007).

Another embodiment the immunological disorder is asthma, the D2S2311 microsatellite allele 402 was found to be a risk allele with OR-2.289, 95% C.I. [1.5443-3.3929] and confirmed in family based studies with χ²=3.96, p=0.0464.

In yet another embodiment, the D2S2311 microsatellite allele 400 was found to be a protective allele with Odds Ratio 0.575, 95% C.I. [0.4098-0.8068] and confirmed in family based studies with χ²=11.306, p=0.0008.

Another embodiment to the present invention, the CA repeat allelic variants at locus M3 have been found to be associated with susceptibility to asthma χ²=11.467334, p=0.000600 and confirmed in family based studies (p=0.008).

Another embodiment to the present invention, the CA repeat allelic variant 154 at locus M3 has a frequency of 81% in patients.

Still another embodiment relates to the CA repeat at locus M3 wherein allelic variant 152 has a frequency of 19% in patients.

In another embodiment to the present invention, the CA repeat allelic variant 154 has been found to be a risk allele in case-control study with OR 1.734, 95% C.I. [1.249-2.407]. This observation was also confirmed in family based study χ2=7.078, p value=0.008.

Yet another embodiment relates to +92031 A/T polymorphism at S1 locus wherein allele T at the S1 locus has frequency of 19% in patients.

Yet another embodiment relates to +92031 A/T polymorphism at S1 locus wherein allele A at the S1 locus has frequency of 81% in patients.

Yet another embodiment relates to +92031 A/T polymorphism at S1 locus wherein this polymorphism is associated with susceptibility to asthma with χ²=7.05, p=0.00791 and its confirmation in family based study χ2=9, p value=0.0027.

Another embodiment to the present invention relates to +110832 A/G (rs2278206) functionally important nonsynonymous polymorphism at S4 locus allele wherein allele A was found to be over transmitted to affected offspring in family based study (χ2=11.504, p=0.0007).

Another embodiment to the present invention relates to the +110832 A/G polymorphism causes a threonine to alanine substitution at position 604 in this sequence resulting in a poor PEST sequence, making the protein more stable

Another embodiment to the present invention relates to +131237 C/T polymorphism at S5 locus allele wherein allele T was found to be over transmitted to affected offspring in family based study (χ2=4.545, p=0.03).

Still another embodiment to the present invention relates to the thirty-six novel four-locus haplotypes generated using M1, S1, M3 and S4 loci in case-control study; and the said novel haplotypes are 396_T_{—}154_G, 398_A_{—}152_A, 400_T_{—}152_A, 400_A_{—}152_A, 406_T_{—}152_A, 406_A_{—}156_T, 412_A_{—}154_A, 400_T_{—}154_A, 402_T_{—}152_A, 404_A_{—}156_T, 410_A_{—}154_A, 404_A_{—}152_G, 406_A_{—}152_A, 404_A_{—}152_A, 406_T_{—}152_G, 396_A_{—}152_G, 400_A_{—}154_G, 402_T_{—}154_A, 402_A_{—}152_A, 404_T_{—}154_A, 400_T_{—}154_G, 396_T_{—}152_G, 404_T_{—}152_A, 398_A_{—}152_G, 386_A_{—}154_A, 402_T_{—}152_G, 398_T_{—}152_G, 404_A_{—}154_G, 408_A_{—}154_A, 406_A_{—}154_A, 398_A_{—}154_A, 404_T_{—}152_G, 400_A_{—}154_A, 400_T_{—}152_G, 402_A_{—}154_A, 404_A_{—}154_A.

Still another embodiment to the present invention relates to the confirmation of twenty-eight novel four-locus haplotypes in families namely, 396_T_{—}154_G, 398_A_{—}152_A, 400_T_{—}152_A, 400_A_{—}152_A, 406_T_{—}152_A, 406_A_{—}156_T, 412_A_{—}154_A, 400_T_{—}154_A, 402_T_{—}152_A, 404_A_{—}156_T, 410_A_{—}154_A, 404_A_{—}152_G, 406_A_{—}152_A, 404_A_{—}152_A, 406_T_{—}152_G, 396_A_{—}152_G, 400_A_{—}154_G, 402_T_{—}154_A, 402_A_{—}152_A, 404_T_{—}154_A, 400_T_{—}154_G, 396_T_{—}152_G, 404_T_{—}152_A, 398_A_{—}152_G, 386_A_{—}154_A, 402_T_{—}152_G, 398_T_{—}152_G, 404_A_{—}154_G, 408_A_{—}154_A, 406_A_{—}154_A, 398_A_{—}154_A, 404_T_{—}152_G, 400_A_{—}154_A, 400_T_{—}152_G, 402_A_{—}154_A, 404_A_{—}154_A

In yet another embodiment to the invention, novel haplotype 402_A_{—}154_A comprising loci M1_ S1_ M3 _S4 has percentage frequency 16.14% in patients in case-control study.

In still another embodiment to the present invention, the haplotype 402_A_{—}154_A was strongly associated with occurrence of asthma (odds ratio 3.68 with 95% CI: 2.2977, 5.916, p value<0.0001) indicating high risk in case-control study.

In yet another embodiment to the present invention, the risk haplotype 402_A_{—}154_A was also found to be over-transmitted to affected offspring in family based study (χ2=4.2714, DF=1, p value=0.038).

In yet another embodiment to the invention, novel haplotypes of loci M1_ S1_ M3 _S4 is 400_A_{—}154_A with percentage frequency of 7.29% in patients.

In yet another embodiment to the present invention, the haplotype 400_T_{—}152_G and 400_A_{—}154_A were negatively associated with occurrence of atopic asthma indicating protective haplotypes.

Yet another embodiment to the present invention, relates to the confirmation of negative association of 400_T_{—}152_G haplotype with occurrence of atopic asthma in family based study (χ²=8.065, DF=1, p value=0.0045).

Yet another embodiment to the present invention, relates to the identification of novel splice variants in atopic asthmatic subjects.

In an embodiment to the invention INPP4A protein expression is reduced in ovalbumin sensitized and challenged mice than in the saline treated controls and its restoration after treatment with known anti-inflammatory steroid agent, such as dexamethasone.

Yet another embodiment to the invention relates to a diagnostic kit comprising at least one specific oligonucleotide pair. Optional additional components of the kit include, for example, restriction enzymes, reverse-transcriptase or polymerase and the substrate.

Detailed Methodology

Isolation of Genomic DNA from Peripheral Blood Leukocytes of the Atopic Asthmatic Patients and the Normal Control Individuals:

Genomic DNA was isolated from the peripheral blood of the patients and control individuals using a modified salting out procedure (Nagarkatti R et al., 2002). Briefly, 10 ml blood was obtained from patients and unrelated control individuals using ACD Vaccutainers (BD Biosciences, San Jose, Calif., USA). Equal volume of ice cold C1 buffer (4×) was added and then 30 ml of ice cold sterile water was added to cause cell membrane lysis (Promega Genomic DNA Isolation Handbook). Following this, the nuclei were pelleted at 1300×g for 15 min at 4° C. The pellet was washed again with 1× C1 buffer. 12 ml of nuclear lysis buffer was added with 0.8ml of 10% SDS. 50 μl of a 20 μg/μl solution of proteinase-K was added and the pellet resuspended by brief vortexing. After incubation at 65° C. for 2-3 hrs, the proteinaceous material was precipitated with the addition of 4 ml of 6M NaCl. After centrifugation for 15 min at 2500 rpm, the supernatant was transferred to another tube and two volumes of absolute ethanol (at room temperature) was used to precipitate the DNA (Miller et al., 1988). The precipitated DNA was then washed with 70% ethanol twice, air-dried, and dissolved in TE buffer. Appropriate dilutions (1:100, in T.E buffer) were used to determine the OD at 260 nm and 280 nm. DNA quality was assessed using the 260 nm/280 nm ratio. The stock solution of the DNA was diluted to 50 ng/μl and used for PCR amplification and genotyping experiments. The stock DNA solution was stored at −20° C.

Identification of Putative Repeats in and Around the INPP4A Gene Using RepeatMasker™ Software:

Besides two known microsatellites, namely D2S2311, D2S2187, five repetitive sequences were identified in the INPP4A gene in the study population using RepeatMasker™ Software, but only CT repeat in intron 11 was found to be polymorphic and studied in detail (FIG. 1B).

Designing and Synthesis of Oligonucleotide Primers for PCR Amplification of INPP4A Gene Variants:

Primers were designed using DNASTAR Primer Select Software SEQ ID: 7, 8, 9, 10, 11, 12, 13, 14, 15, 16, 17, 18, 19, 20, 21, 22, 23, 25 and 26. The reverse primers for the repeat polymorphisms were labeled with 6-FAM for detection by fragment analysis on 3100 automated capillary array sequencer.

PCR Amplification Conditions for Different Primer Sets:

PCR Amplification of SEQ ID #1 and SEQ ID #2:

Primers: SEQ ID #7, 8 and SEQ ID #9, 10

PCR amplification of genomic DNA samples isolated from peripheral blood leukocytes of the atopic asthmatic patients and normal control individuals using the above said primers in a pooled reaction. PCR was carried out in a total volume of 5 μl containing 25 ng of genomic DNA, 1.0 pmol each of 6-FAM-labeled reverse primers and non-labeled forward primers, 1.5 Mm MgCl₂, 0.25 mM of each dNTP, 0.03 U/μl of Taq DNA polymerase (Bangalore Genie, India) and the buffer recommended by the supplier. After PCR, 1 μl of the PCR product was loaded with and internal size standard (PET labeled) on ABI Prism 3100 Genetic Analyzer (Applied Biosystems). Fragment lengths were determined using the (Genotyper 3.7, Applied Biosystems).

PCR Conditions:

Denaturation at 94 degree C. for 5 minutes, Thirty five cycles of denaturation at 94 degree C. for 30 seconds, annealing at 65 degree C. for 30 seconds, extension at 72 degree C. for 30 seconds; followed by a 10 minutes 72 degree C. segment extension period.

PCR Amplification of SEQ ID #3:

Primer: SEQ ID #11 and 12

PCR amplification of genomic DNA samples isolated from peripheral blood leukocytes of the atopic asthmatic patients and normal control individuals using the above said primers. PCR was carried out in a total volume of 5μl containing 25 ng of genomic DNA, 1.0 pmol each of a 6-FAM-labeled forward primer and a non-labeled reverse primer, 1.5 Mm MgCl₂, 0.25 mM of each dNTP, 0.03 U/μl of Taq DNA polymerase (Bangalore Genie, India) and the buffer recommended by the supplier. After PCR, 1 μl of the PCR product was loaded with and internal size standard (PET labeled) on ABI Prism 3100 Genetic Analyzer (Applied Biosystems). Fragment lengths were determined using the (Genotyper 3.7, Applied Biosystems).

PCR Conditions:

Denaturation at 94 degree C. for 5 minutes, Thirty five cycles of denaturation at 94 degree C. for 30 seconds, annealing at 68 degree C. for 30 seconds, extension at 72 degree C. for 30 seconds; followed by a 10 minutes 72 degree C. segment extension period.

PCR Amplification of SEQ ID #4:

Primer: SEQ ID #13 and 14

PCR amplification of genomic DNA samples isolated from peripheral blood leukocytes of the atopic asthmatic patients and normal control individuals using the above said primers. PCR was carried out in a total volume of 20 μl containing 50 ng of genomic DNA, 2.0 pmol each of a forward and reverse primer, 1.5 Mm MgCl₂, 0.25 mM of each dNTP, 0.03 U/μl of Taq DNA polymerase (Bangalore Genie, India) and the buffer recommended by the supplier. Purified PCR product was used for Snapshot reaction.

PCR Conditions:

Denaturation at 94 degree C. for 5 minutes, Thirty five cycles of denaturation at 94 degree C. for 45 seconds, annealing at 66 degree C. for 45 seconds, extension at 72 degree C. for 45 seconds; followed by a 10 minutes 72 degree C. segment extension period.

PCR Amplification of SEQ ID #5:

Primer: SEQ ID #15 and 16

PCR amplification of genomic DNA samples isolated from peripheral blood leukocytes of the atopic asthmatic patients and normal control individuals using the above said primers. PCR was carried out in a total volume of 20 μl containing 50 ng of genomic DNA, 2.0 pmol each of a forward and reverse primer, 1.5 Mm MgCl₂, 0.25 mM of each dNTP, 0.03 U/μl of Taq DNA polymerase (Bangalore Genie, India) and the buffer recommended by the supplier. Purified PCR product was used for Snapshot reaction.

PCR Conditions:

Denaturation at 94 degree C. for 5 minutes, Thirty five cycles of denaturation at 94 degree C. for 45 seconds, annealing at 66 degree C. for 45 seconds, extension at 72 degree C. for 45 seconds; followed by a 10 minutes 72 degree C. segment extension period.

PCR Amplification of SEQ ID #6:

Primer: SEQ ID #17 and 18

PCR amplification of genomic DNA samples isolated from peripheral blood leukocytes of the atopic asthmatic patients and normal control individuals using the above said primers. PCR was carried out in a total volume of 20 μl containing 50 ng of genomic DNA, 2.0 pmol each of a forward and reverse primer, 1.5 Mm MgCl₂, 0.25 mM of each dNTP, 0.03 U/μl of Taq DNA polymerase (Bangalore Genie, India) and the buffer recommended by the supplier. Purified PCR product was used for Snapshot reaction.

PCR Conditions:

Denaturation at 94 degree C. for 5 minutes, Thirty five cycles of denaturation at 94 degree C. for 45 seconds, annealing at 68 degree C. for 45 seconds, extension at 72 degree C. for 45 seconds; followed by a 10 minutes 72 degree C. segment extension period.

Direct Sequencing of the Purified PCR Products

Direct sequencing of the purified PCR products using dye terminator chemistry was carried out on an ABI Prism 3100 automated DNA sequencer for gene segments with SEQ ID #4, 5 and 6. Sequencing was carried out using specific primers viz: 13, 14, 15, 16, 17 and 18 on an ABI 3100 capillary sequencer (Applied Biosystems, Foster City, Calif., USA) for a minimum of 20 atopic asthmatic and 20 control individuals. PCR product was gel purified for sequencing. Briefly, sequencing primers, diluted to 1 pmol per μl, and 75-150 ng/μl PCR product were added to 5 μl reaction mix, and volume made up to 10 μl with autoclaved MilliQ water as per the Big Dye Terminator kit instructions (Applied Biosystems, Foster City, Calif., USA). PCR was set up with the following conditions: 96° C. for 5 seconds, 55° C. for 30 seconds and 60° C. for 4 minutes. Sequencing reactions were purified with 70% ethanol washes to remove unincorporated primers and fluorescent ddNTPs. Briefly, 26μl autoclaved MilliQ water was added to the sequencing reaction. Sixty-four microliters of chilled 100% ethanol was added to the tubes and vortexed. The tubes were centrifuged at 16,000 g for 20 minutes at room temperature. Washes were performed with 70% ethanol by centrifugation at 16,000 g for 5 minutes. The pellets were air dried and resuspended in 10 μl of 100% Hi-Di formamide. The tubes were incubated at 94° C. for 5 minutes and placed in the 3100 Automated Sequencer. Sequence analysis was carried out using Sequence Navigator (ver 2.1, Applied Biosystems, Foster City, Calif., USA) and DNAStar (ver 1.1, DNASTAR) software. Homozygous and heterozygous alleles were scored manually.

Genotyping of +92031 A/T, +92344 C/T, +92817 C/T, +110832 A/G and +131237 C/T Polymorphisms:

The +92031 A/T, +92344 C/T, +92817 C/T, +110832 A/G and +131237 C/T polymorphisms were selected on the basis of linkage disequilibrium among all the SNPs (FIG. 2) and studied using SNaPshot. ddNTP Primer Extension Kit (Applied Biosystems, Foster City, USA). SnaPshot PCR was carried out using 50 ng purified PCR template, 1 pmol primer with SEQ Ids 19, 20, 21, 22 and 23 resp. and ABI ready reaction mix and 1× dilution buffer (as supplied by the manufacturer). PCR was set up with the following conditions: 96° C. for 10 seconds, 58° C. for 5 seconds and 60° C. for 30 seconds for a total of 30 cycles. To clean up the primer extension reaction, 1 U of calf intestinal phosphatase (CIP) diluted in 10× NEB3 (New England Biolabs), was added to the reaction mixture and the mixture was incubated at 37° C. for 1 hour, followed by an incubation for 15 minutes at 72° C. for enzyme inactivation. These samples were subsequently electrophoresed using the ABI Prism 3100 Genetic Analyzer as per the manufacturer's instructions. The results were analyzed using the program ABI Prism GeneScan™ and Genotyper™ (Applied Biosystems, Foster City, USA).

Functional Significance of +110832 A/G (rs2278206) Polymorphism in the Extended Exon 17 Splice Variant α3:

The PEST sequences in the splice variant α3 were identified using PESTFIND tool of ExPASY Proteomic database (www.at.embnt.org/embnet/tools/bio/pestfind/). The protein sequence was downloaded from NCBI (AAK58870). The 977 amino acid splice form of protein contains additional amino acids from extended exon 17. The +110832 A to G polymorphism results in a nonsynonymous threonine to alanine substitution at position 604 in the amino acid sequence. The amino acid sequence was changed at 604 position from threonine to alanine and this sequence was again run through PESTFIND for finding PEST sequences. Platelets were isolated from peripheral blood of normal healthy individuals having AA, GG and AG genotypes for A+110832G polymorphism and stimulated with 2 μM ionomycin in platelet suspension buffer for 5 and 10 minutes, respectively. Western blot was performed using goat polyclonal antibody sc-12315 and anti-goat HRP antibody. Densitometry scanning of the bands was done using Alphalmager (Alpha Innotech Corporation, San Leandro, Calif.) (FIG. 7).

Identification of Novel Splice Variants in the Exon 15-Exon 19 Region of INPP4A Using Primers with SEQ ID #25 and 26:

RNA was isolated from the total leukocytes of 12 atopic asthmatics and 6 normal individuals using EZ-RNA isolation kit, following manufacturer's directions (Biological Industries). cDNA was made from 10 μg of total RNA using cDNA Archive kit from Applied Biosystems. PCR was carried out in a total volume of 20 μl containing cDNA corresponding to 100 ng of starting RNA, 2.0 pmol each of forward and reverse primer, 1.5 Mm MgCl₂, 0.25 mM of each dNTP, 0.03 U/μl of Taq DNA polymerase (Bangalore Genie, India) and the buffer recommended by the supplier. PCR conditions:

Denaturation at 94 degree C. for 5 minutes, Thirty cycles of denaturation at 94 degree C. for 45 seconds, annealing at 65 degree C. for 45 seconds, extension at 72 degree C. for 45 seconds; followed by a 10 minutes 72 degree C. segment extension period.

The 598 base pair splice variant (FIG. 8) was sequenced as described above and the deletion of exon 16 was was confirmed.

Calculating and Estimating the Frequency of Repeat Polymorphisms D2S2311, D2S2187 and That of +99095 CA, Single Nucleotide Polymorphisms +92031 A/T, +92344 C/T, +92817 C/T, +110832 A/G and +131237 C/T:

CLUMP software with Monte Carlo simulations has been used to test the allelic association of for multi-allelic markers with disease phenotype for repeat polymorphisms. Odds ratios were calculated and Chi-square tests were performed to study the association with disease phenotype. The repeats have been denoted according to the fragment length where sizing was done using internal size standard during gene scan. SNPs are designated as S1, S2, S3, S4 and S5.

Estimating the Frequencies of Haplotypes Generated Using Four Loci in the Normal Individuals and Atopic Asthmatic Patients for Finding Association Between These Haplotypes and the Disease:

Novel haplotypes for loci M1, S1, M3 and S4, have been generated using the PHASE program for the patient (N=192) and control (N=272) groups (Stephens M, Am J Hum Genet. Nov 73:1162-9, 2003). Default parameters with 100 iterations were used to generate the haplotypes (http://archimedes.well.ox.ac.uk/pise/PHASE-simple.html, PHASE Ver. 2.0.2). Chi-squares and Odds ratios were calculated for association with phenotype.

Transmission Disequilibrium (TDT) Analysis of Repeat Polymorphisms D2S2311, D2S2187 and That of +99095 CA Single Nucleotide Polymorphisms +92031 A/T, +92344 C/T, +92817 C/T, +110832 A/G and +131237 C/T and Their Haplotypic Analysis in Nuclear Families:

In the families, allele-wise TDT analysis was done using TDT-sTDT (http://genomics.med.upenn.edu/spielman/TDT.htm). Haplotypic transmission to affected individual was observed using TRANSMIT (Transmit, version 2.5.4).

So the matter in which the above-mentioned features, advantages and the objects of the invention, as well as others, which will become clear, are attained and can be understood in detail. These drawings form a part of the specification. It is to be noted, however, that the appended drawings illustrate preferred embodiments of the invention and therefore not to be considered limiting in their scope.

Developing Ovalbumin Sensitized and Challenged Mouse Model of Asthma and Saline Treated Control Mice:

The BALB/c mice were sensitized by intra-peritoneal injections of 20 μg chicken egg ovalbumin (OVA) (grade V≧98% pure, Sigma Chemical Co., St. Louis, Mo., USA) in 0.2 ml saline adsorbed on 2 mg alum (sensitized) or alum alone (control) on days 0, 7 and 14. Mice were then challenged with aerosolized 3% ovalbumin (sensitized) or aerosolized saline (control) 30 minutes per day for 10 consecutive days (from day 22 to day 32). Responsiveness to methacholine (Sigma-Aldrich) was assessed in conscious, unrestrained mice by barometric plethysmography, using protocol, apparatus and software supplied by BUXCO (Troy, N.Y., USA). Mice were sacrificed 16 hrs after the last challenge and lung tissue were taken for immunohistochemistry.

Treatment of Ovalbumin Induced Mouse Model of Asthma with Steroid:

The BALB/c mice were sensitized by intra-peritoneal injections of 20 μg chicken egg ovalbumin (OVA) (grade V≧98% pure, Sigma Chemical Co., St. Louis, Mo., USA) in 0.2 ml saline adsorbed on 2 mg alum on days 0, 7 and 14. Mice were then challenged with aerosolized 3% ovalbumin 30 minutes per day for 10 consecutive days (from day 22 to day 32). Treatment with 1 mg/kg dexamethasone was given starting from the day of last sensitization to last challenge (day 14-32). Bronchial hyperresponsiveness was measured as described previously using BUXCO plethysmograph (Troy, N.Y., USA). Mice were sacrificed 16 hrs after the last OVA challenge and lungs were subjected to immunohistochemistry.

Immunohistochemistry of Mouse Lung Tissue Section for INPP4A Protein:

Immunohistochemistry of mouse lung section was done using commercial goat polyclonal antibody sc-12315 (Santa Cruz Biotechnology, Inc.; CA, USA), raised against a peptide near the amino terminus of INPP4A of human and mouse origin following standard protocol with slight modifications (Ather M H et al, 2004). Goat gamma globulin was used as isotype control (Jackson Immunoresearch Laboratories, Inc.; PA, USA).

### BRIEF DESCRIPTION OF THE ACCOMPANYING DRAWINGS

FIG. 1A shows the schematic presentation of the different known spliceoforms of INPP4A, FIG. 1B shows a schematic presentation of the different polymorphic regions of the INPP4A gene studied. The D2S2311 repeat, 44.7 kb upstream of the promoter, is denoted as M1. The D2S2187 repeat is referred to as M2, the CA polymorphism at is denoted as M3. All five of the polymorphisms are also shown in sequence context below the gene.

FIG. 2 shows graphical overview of linkage disequilibrium for the eight loci studied using GOLD.

FIG. 3 shows the distribution of D2S2311 alleles in patient and control group. The figure depicts the allele frequencies at the D2S2311 repeat locus with the fragment sizes depicted on the X-axis and their respective frequencies on the Y-axis.

FIG. 4 shows allelic and genotypic distribution of +92031 A/T (S1) in case-control study.

FIG. 5 shows allelic and genotypic distribution of CA repeat polymorphism in intron 11 in case-control study.

FIG. 6 shows the distribution of haplotypes (M1_S1_M3_S4) in Patients and Controls (Np=192, Nc=272) (Table 2). The figure depicts the haplotype frequencies on the X-axis and their respective frequencies on the Y-axis.

FIG. 7 shows the potential PEST sequence and its experimental validation in the extended exon 17 splice variant region and comparison with Thr to Ala substitution variant.

FIG. 8 shows the gel picture of known α3 splice variant along with the novel splice variants identified in atopic asthmatic patients.

FIG. 9 shows the immunohistochemistry of mouse lung sections from saline treated control mice, ovalbumin sensitized mice and the steroid treated ovalbumin sensitized and challenged mice.

### ASSOCIATION OF THE REPEAT LOCI WITH ATOPIC DISORDERS SUCH AS ASTHMA

To demonstrate the association of the M1 and M2 and M3 repeat locus with atopic disorders such as asthma, chi square test was performed using CLUMP software. CLUMP is a program designed to assess the significance of the departure of observed values in a contingency table from the expected values conditional on the marginal totals. The significance is assessed using a Monte Carlo approach, by performing repeated simulations to generate tables having the same marginal totals as the one under consideration, and counting the number of times that a chi-squared value associated with the real table is achieved by the randomly simulated data. This means that the significance levels assigned should be unbiased (with accuracy dependent on the number of simulations performed) and that no special account needs to be taken of continuity corrections or small expected values. The method is described in full in: Sham PC & Curtis D.1995. Monte Carlo tests for associations between disease and alleles at highly polymorphic loci. Ann Hum Genet. 59: 97-105. A significantly different pattern of distribution of the alleles between the two groups was obtained; for alleles at M1 locus and found to be associated with susceptibility to asthma X2=43.441128, DF=9, p value<0.0001. Allele 402 was found to be a risk allele with Odds Ratio 2.289, 95% C.I. [1.5443-3.3929] allele 400 was found to be a protective allele with Odds Ratio 0.575, 95% C.I. [0.4098-0.8068] as calculated by 2×2 table (http://home.clara.net/sisa/twoby2.htm) (FIG. 3). Similar results were obtained in family based association studies (p value=0.0007) (Table 3a).

There was no significantly different pattern of distribution of the alleles between the two groups for M2 (p=0.214179) in case-controls (Table 3b).

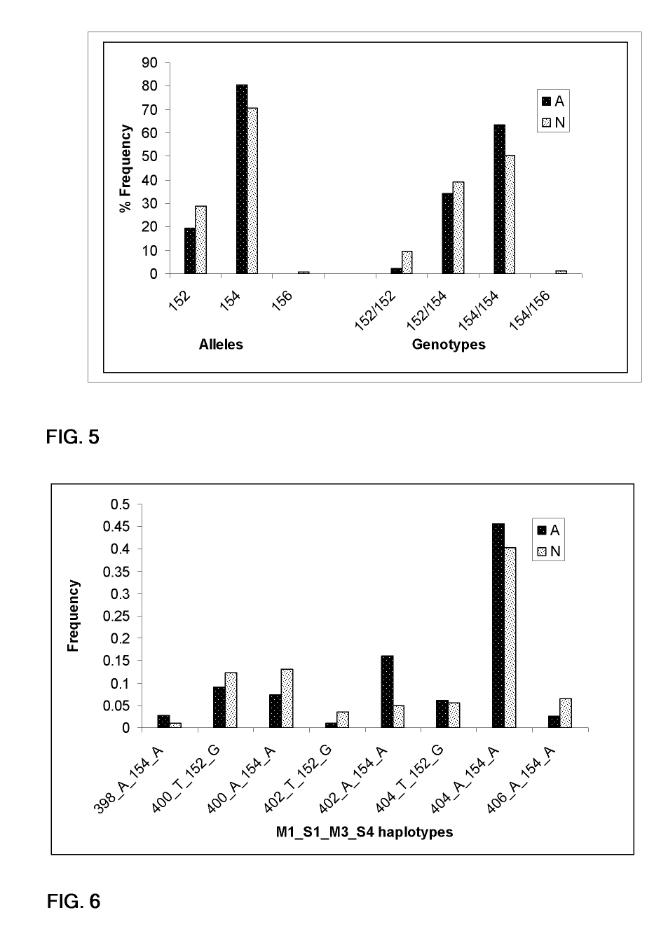

The locus M3 was also found to be significantly associated with asthma (p=0.0006). The CA repeat allelic variants at locus M3 have been found to be associated with susceptibility to asthma χ²=11.467334, p=0.0006. The CA repeat allelic variant 154 has been found to be a risk allele in case-control study with OR 1.734, 95% C.I. [1.249-2.407] (FIG. 5). This observation was also confirmed in family based study χ2=7.078, p value=0.008 (Table 3f).

Demonstration of Association of +92031 A/T (S1) Polymorphism with Asthma:

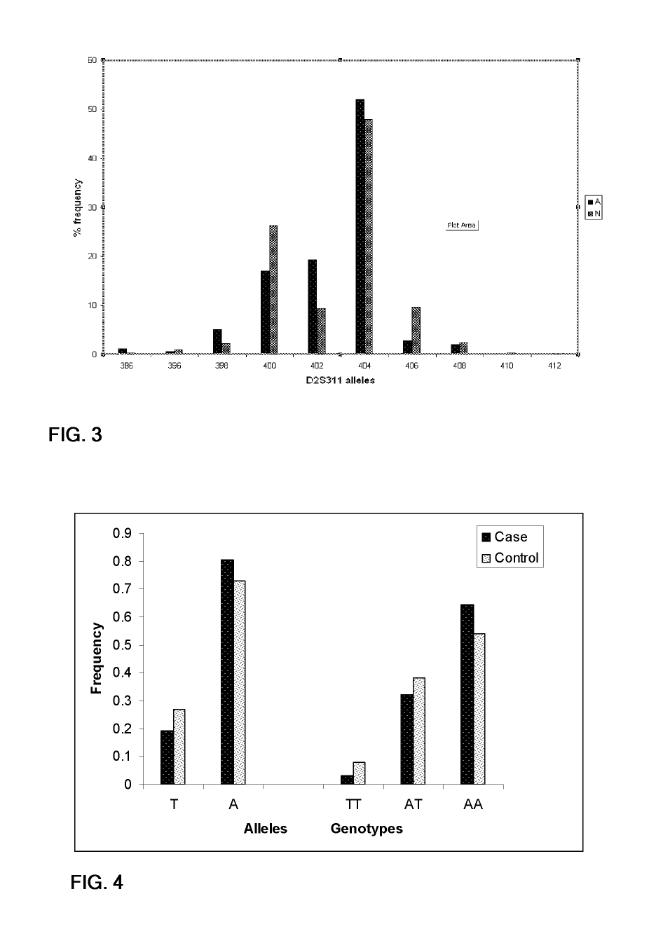

To demonstrate the association of +92031 A/T polymorphism, Armitage trend test was performed. The pattern of distribution of the three genotypes, AA, AT and TT was significantly different in the two groups studied (χ2=7.05, p=0.008). The A allele was predominant in the cases as compared to the control group {OR=1.542, 95% C.I. (1.121-2.120), p=0.0075} (FIG. 4). Similarly, the T allele was found to be over transmitted to affected offsprings in family based study using TDT-sTDT program (χ2=9, p=0.0027) (Table 3c).

Demonstration of Association of +110832 A/G (S4) Polymorphism with Asthma:

To demonstrate the association of +110832 A/G nonsynonymous polymorphism, Armitage trend test was performed in case-control study. The risk allele T showed a marginal association with the disease phenotype {Odds_ratio=1.324, 95% C.I. (0.964-1.819), χ2=3.01, p=0.08). The A allele was found to be over transmitted to affected offsprings in family based study using TDT-sTDT program (χ2=11.5, p=0.0007) (Table 3g).

Generation of Haplotypes:

We then used the PHASE program to generate haplotypes for the patient and control groups. The program PHASE implements a new statistical method for reconstructing haplotypes from population genotype data. Experiments with the software on both real and simulated data indicate that it can provide an improvement on the EM algorithm for reconstructing haplotypes. It allows for missing genotype data and also can handle more than one locus irrespective of the polymorphism, for example SNPs and repeats can be analyzed simultaneously. Based on the output from the software the probability values of the haplotypes are also predicted and can be utilized to differentiate more confident haplotypes. The PHASE software is suitable for genetic distances of 100 cM or less. Similary family based haplotypic analysis was performed using TRANSMIT program. TRANSMIT tests for association between genetic marker and disease by examining the transmission of markers from parents to affected offspring. The tests are based on a score vector which is averaged over all possible configurations of parental haplotypes and transmissions consistent with the observed data.

In case-control study, the haplotypes whose expected frequency was larger than 0.025, in either of the two groups are shown in Table 2 (FIG. 6). The odds in favor of patients rather than controls having 402_A_{—}154_A haplotype were 3.68 with 95% CI: 2.2977, 5.916. The corresponding likelihood ratio χ2 tests showed p-value less than 10-5. TRANSMIT also showed similar results in family based association study (χ2=4.2714, DF=1, p value=0.038). Thus the 4-locus haplotype, 402_A_{—}154_A was strongly associated with asthma. On the other hand, the odds in favor of patients rather than controls having haplotype 400_T_{—}152_G and 400_A_{—}154_A were 0.16, 95% CI: 0.46-1.09 and 0.12, 95% CI: 0.33-0.82 respectively. The haplotype 400_T_{—}152_G was also found to be negatively associated with occurrence of atopic asthma in family based study. (χ²=8.065, DF=1, p value=0.0045). Thus, haplotypes 402_A_{—}154_A and 400_T_{—}152_G were identified to be major risk and protective haplotypes respectively.

Functional Significance of +110832 A/G (rs2278206) Polymorphism in the Extended Exon 17 Splice Variant α3:

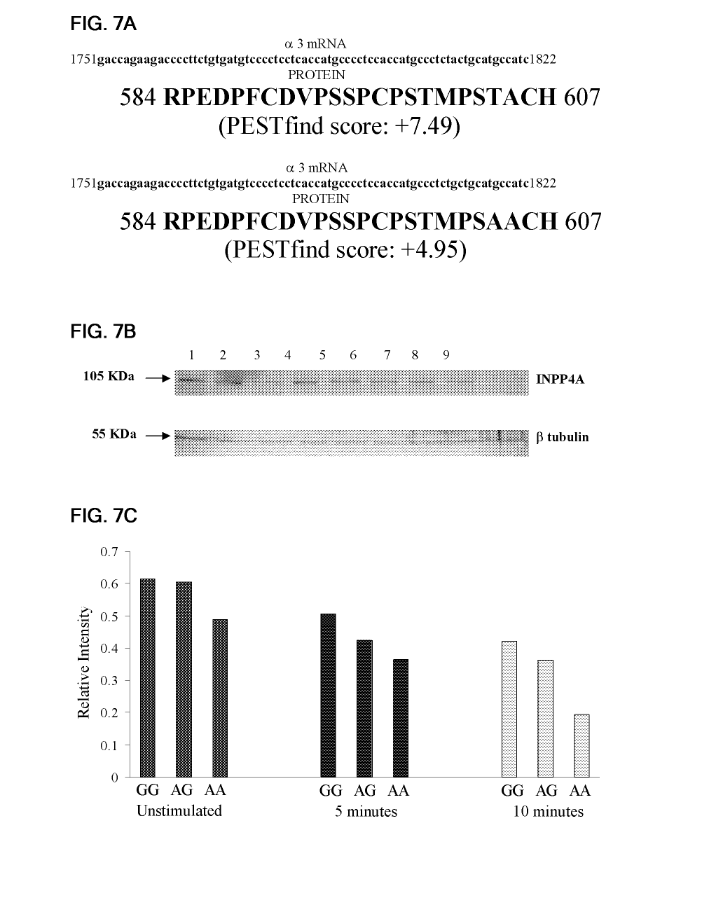

Threonine to Alanine substitution at amino acid 604 in the α3 splice variant of INPP4A gene resulted in a change in the PESTfind score from +7.49 to +4.95 making it a poor PEST sequence. Protein from platelets of individuals with AA genotype was found to be more susceptible to degradation as seen by nearly 60% degradation within 10 min of stimulation whereas the protein from individuals with GG genotype was less susceptible (nearly 31% degradation). Also, the basal levels of INPP4A protein from individuals with AA genotypes was found to be lower (data not shown), suggesting poor stability in vivo as well (FIG. 7).

Identification of Novel Splice Variants in the Exon 15-Exon 19 Region of INPP4A:

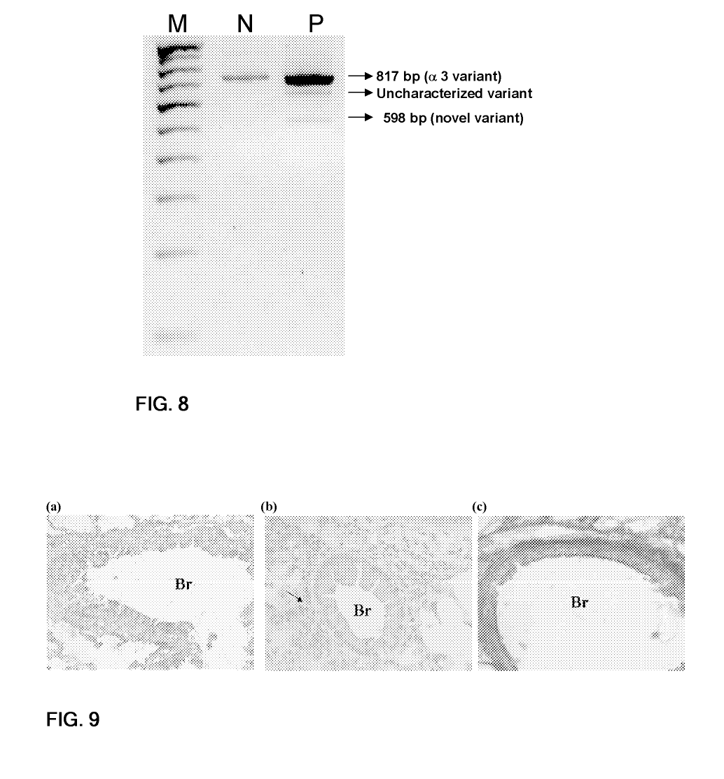

Novel splice variant of 598 base pair was identified in asthmatic patients. This variant has deleted exon 16 (219 bases) as confirmed by sequencing (FIG. 8).

Significant Decrease in the INPP4A Protein Expression in Allergen-Induced Mouse Model of Asthma:

The INPP4A protein was found to be expressed at significantly lower levels in ovalbumin induced mouse model of asthma as compared to saline treated controls (FIG. 9).

Restoration of INPP4A Protein in Ovalbumin Induced Mice by Treatment with Anti-Inflammatory Agent:

The levels of INPP4A protein were found to be restored in case of steroid treated ovalbumin induced mouse model of asthma. Noticeably, the expression of INPP4A protein after steroid treatment was found to be even higher than the saline treated control mice (FIG. 9).

Analysis of Polymorphisms:

A. Preparation of Samples:

Polymorphisms are detected in a target nucleic acid from an individual being analyzed. For assay of genomic DNA, virtually any biological sample (other than pure red blood cells) is suitable. For example, convenient tissue samples include whole blood, semen, saliva, tears, urine, fecal material, sweat, buccal, skin and hair. For assay of cDNA or mRNA, the tissue sample must be obtained from an organ in which the target nucleic acid is expressed.

Many of the methods described below require amplification of DNA from target samples. This can be accomplished by e.g., PCR. See generally PCR Technology: Principles and Applications for DNA Amplification (ed. H. A. Erlich, Freeman Press, N.Y., N.Y., 1992); PCR Protocols: A Guide to Methods and Applications (eds. Innis, et al., Academic Press, San Diego, Calif.,1990); Mattila et al., Nucleic Acids Res. 19, 4967 (1991) and U.S. Pat. No. 4,683,202 (each of which is incorporated by reference for all purposes).

Other suitable amplification methods include the Ligase Chain Reaction (LCR) (see Barringer K J et al, Gene 89:117-22, 1990; Friedhoff P et al, Anal Biochem 215:9-16, 1993) and Nucleic Acid Based Sequence Amplification (NASBA). The latter two amplification methods involve isothermal reactions based on isothermal transcription, which produce both single stranded RNA (ssRNA) and double stranded DNA (dsDNA) as the amplification products in a ratio of about 30 or 100 to 1, respectively.

B. Detection of Polymorphisms in Target DNA:

There are two distinct types of analysis depending on whether a polymorphism in question has already been characterized or not. The first type of analysis is sometimes referred to as de novo characterization. This analysis compares target sequences in different individuals to identify points of variation, i.e., polymorphic sites. By analyzing groups of individuals representing the greatest ethnic diversity among humans and greatest breed and species variety in plants and animals, patterns characteristic of the most common alleles/haplotypes of the locus can be identified, and the frequencies of such populations in the population determined. Additional allelic frequencies can be determined for subpopulations characterized by criteria such as geography, race, or gender. The de novo identification of the polymorphisms of the invention is described in the Examples section. The second type of analysis is determining which form(s) of a characterized polymorphism are present in individuals under test. There are a variety of suitable procedures, which are discussed in turn.

1. Repeat Detection (Size Variation Detection):

The design and use of primers flanking the sequence contain the repeat sequence or other polymorphic elements, which lead to a size difference. PCR amplification of the sequence leads to the presence of a pool of amplified products that differ by the specific repeat or polymorphism size. These size differences can then be detected using gel based, charge based methods. Usually for gel-based detection one of the primers is labeled with a fluorescent compound which can then be excited and detected using a CCD camera or other methods.

2. Allele-Specific Probes:

The design and use of allele-specific probes for analyzing polymorphisms is described by e.g., Saiki et al., Nature 324, 163-166, 1986; Dattagupta, EP 235,726, Saiki, WO 89/11548. Allele-specific probes can be designed that hybridize to a segment of target DNA from one individual but do not hybridize to the corresponding segment from another individual due to the presence of different polymorphic forms in the respective segments from the two individuals.

3. Allele-Specific Primers:

An allele-specific primer hybridizes to a site on target DNA overlapping a polymorphism and only primes amplification of an allelic form to which the primer exhibits perfect complementarity. This primer is used in conjunction with a second primer which hybridizes at a distal site. See, e.g., WO 93/22456.

4. Direct-Sequencing:

The direct analysis of the sequence of polymorphisms of the present invention can be accomplished using either the dideoxy chain termination method or the Maxam Gilbert method (see Sambrook et al., Molecular Cloning, A Laboratory Manual (2nd Ed., CSHP, New York 1989); Zyskind et al., Recombinant DNA Laboratory Manual, (Acad. Press, 1988)).

5. Denaturing Gradient Gel Electrophoresis:

Amplification products generated using the polymerase chain reaction can be analyzed by the use of denaturing gradient gel electrophoresis. Different alleles can be identified based on the different sequence-dependent melting properties and electrophoretic migration of DNA in solution. Erlich, ed., PCR Technology, Principles and Applications for DNA Amplification, (W.H. Freeman and Co, New York, 1992), Chapter 7.

6. Single-Strand Conformation Polymorphism Analysis:

Alleles of target sequences can be differentiated using single-strand conformation polymorphism analysis, which identifies base differences by alteration in electrophoretic migration of single stranded PCR products, as described in Orita et al., Proc. Nat. Acad. Sci. 86, 2766-2770, 1989. Amplified PCR products can be generated as described above, and heated or otherwise denatured, to form single stranded amplification products. Single-stranded nucleic acids may refold or form secondary structures which are partially dependent on the base sequence. The different electrophoretic mobilities of single-stranded amplification products can be related to base-sequence difference between alleles of target sequences.

Methods of Use:

After determining polymorphic form(s) present in an individual at one or more polymorphic sites, this information can be used in a number of methods.

A. Correlation of Polymorphisms with Phenotypic Traits:

Atopic diseases are heterogeneous in nature and as such there are many sub-phenotypes and traits to which the association can be observed. The polymorphisms of the invention may contribute to the phenotype of an organism in different ways. As described above, the polymorphisms may act at various levels of cellular organization by which the disease phenotypes are observed as the end result. These polymorphisms may yield different selection advantages or disadvantages. For example, a heterozygous sickle cell mutation confers resistance to malaria, but a homozygous sickle cell mutation is usually lethal. A single polymorphism may affect more than one phenotypic trait.

Likewise, a single phenotypic trait may be affected by polymorphisms in different genes. Further, some polymorphisms predispose an individual to a distinct mutation that is causally related to a certain phenotype. Phenotypic traits include diseases that have known but hitherto unmapped genetic components. Phenotypic traits also include symptoms of, or susceptibility to, multifactorial diseases of which a component is or may be genetic, such as atopy, autoimmune diseases, inflammation, cancer, diseases of the nervous system, and infection by pathogenic microorganisms. Some examples of autoimmune diseases include systemic lupus erythematosus, rheumatoid arthritis, diabetes, multiple sclerosis, (insulin-dependent and non-independent), and Graves disease. Some examples of cancers include cancers of the breast, bladder, colon, brain, etc. As such, phenotypic traits also include characteristics, for example, susceptibility or receptivity to particular drugs or therapeutic treatments.

To perform association analysis of the disease phenotypes and genetic markers, the presence or absence of a set of polymorphisms (i.e. a polymorphic set) is determined for a set/population of the individuals, some of whom exhibit a particular trait termed variously as case/patients/affected/diseased individuals etc, and some of which exhibit lack of the trait termed variously as control individuals/normal etc. The alleles of each polymorphism of the set are then counted to determine if the presence or absence of a particular allele or a set of alleles or a haplotype is associated with the trait of interest. Test for such associations can be performed by standard statistical methods such as a χ2 test etc. Based on the values obtained for the hypothesis tested for example, if the allele X is present more in patients than in controls and if the allele X is not present more in patients than in controls, the significance value is obtained. If this value lies in a particular range then it determines the significance level of the correlations. For example, it might be found that the presence of allele A1 at polymorphic site 1 correlates with cystic fibrosis disease. As a further example, it might be found that the combined presence of allele A1 at polymorphic site 1 and allele B1 at polymorphic site 2 correlates with 10 fold-increased severity of cystic fibrosis.

Such associations can be of immediate benefit if an extremely strong correlation exists. For example, detection of cystic fibrosis polymorphism A1 and B1 in a patient may allow for rapid diagnosis and discrimination from other diseases which exhibit similar phenotypes; it can also allow for treatment if available; it can allow for screening of neonates for detection and/or for susceptibility and/or risk assessment; it can allow for selection of better and improved management methods for the disease from those which are available; it may allow for the treatment to be given if it is determined that the polymorphic site also correlates with particular therapeutic regimes and that such therapeutic drugs are more beneficial to the patient than other drugs.

B. Genetic Mapping of Phenotypic Traits:

The previous section concerns identifying correlations between phenotypic traits and polymorphisms that directly or indirectly contribute to those traits. The present section describes identification of a physical linkage between a genetic locus associated with a trait of interest and polymorphic markers that are not associated with the trait, but are in physical proximity with the genetic locus responsible for the trait and co-segregate with it. Such analysis is useful for mapping a genetic locus associated with a phenotypic trait to a chromosomal position, and thereby cloning gene(s) responsible for the trait. Please see (Altshuler D et al, 1998, N Engl J Med 338:1626; Cargill M et al, 1999, Nat Genet 22:231-8; Chang C, 1988, Proc Natl Acad Sci USA 85:6856-60; Hacia J G et al, 1999, Nat Genet 22:164-7; Hirschhorn J N et al, 2000, Proc Natl Acad Sci USA 97:12164-9; Lander E S and Botstein D, 1986, Proc Natl Acad Sci USA 83:7353-7; Lander E S, 1993, Nat Genet 4:5-6; Reich D E et al, 2001, Nature 411:199-204; Sachidanandam R et al, 2001, Nature 409:928-33. Genes localized by linkage can be cloned by a process known as directional cloning.

Computer programs are available for the calculation of lod scores for differing values of theta. Other references on linkage and disease mapping use above mentioned approaches include, Kreutz R et al, 1995, Proc Natl Acad Sci USA 92:8778-82; de Gouyon B et al, 1993, Proc Natl Acad Sci USA 90:1877-81; Julier C et al, 1990, Proc Natl Acad Sci USA 87:4585-9; Oberle I et al, 1986, Proc Natl Acad Sci USA 83:1016-20; Lathrop G M et al, 1984, Proc Natl Acad Sci USA 81:3443-6; Cohen D et al, 1984, Proc Natl Acad Sci USA 81:1774-8.

Modified Polypeptides and Gene Sequences

The invention further provides variant forms of nucleic acids. These variants can be used to identify the chromosomal backgrounds of individuals and depending on the particular haplotype, risk may be assessed. The promoter polymorphism may also be important in the production of variant gene constructs containing the gene of interest so as to allow heterologus expression of the gene in various human and non-human cell lines. 5′-UTR polymorphism may lead to variant expression level changes due to transcriptional or post translational modifications.

Kits

The invention further provides kits comprising at least one specific oligonucleotide pair. For example, the same substrate can be used as a template for allele-specific oligonucleotide probes for detecting all of the polymorphisms listed. For initial screening purposes, the D2S2311 repeat polymorphism found 44.7 kb upstream of the human INPP4A gene could be useful as the allele 400 of this polymorphism is negatively associated, whereas the allele 402 is positively associated with asthma. For this locus, PCR was carried out in a total volume of 5 μl containing 25 ng of genomic DNA, 1.25 pmol each of a 6-FAM-labelled forward primer and a non-labeled reverse primer, 1.5 Mm MgCl₂, 0.25 mM of each dNTP, 0.03 U/μl of Taq DNA polymerase (Bangalore Genie, India) and the buffer recommended by the supplier. After PCR, 1 μl of the PCR product was loaded with an internal size standard (PET labeled) on ABI Prism 3100 Genetic Analyzer (Applied Biosystems). Fragment lengths were determined using the Genotyper Software version 3.7 (Applied Biosystems). If subsequently required, genotyping at the other seven loci, namely, +92031 A/T, +92344 C/T, +92817 C/T, +110832 A/G, +131237 C/T and +99095 CA repeat could also be carried out.

Additionally, the kit also provides oligonucleotide pairs for detecting novel splice variants of the INPP4A mRNA. Optional additional components of the kit include, for example, restriction enzymes, reverse-transcriptase or polymerase, the substrate nucleoside triphosphates, means used to label (for example, an avidin-enzyme conjugate and enzyme substrate and chromogen if the label is biotin), and the appropriate buffers for reverse transcription, PCR, or hybridization reactions. Usually, the kit also contains instructions for carrying out the methods.

The following examples are given by way of illustration only and therefore should not be construed to limit the scope of the present invention.

### EXAMPLE 1

Association of D2S2311 Repeat Locus with Atopic Disorders Such as Asthma:

The 401 bp DNA stretch of SEQ ID No. 1 of INPP4A gene having the D2S2311 repeat polymorphism was PCR amplified using novel primers of SEQ ID No. 7 and 8. PCR amplification of genomic DNA samples isolated from peripheral blood leukocytes of the atopic asthmatic patients and normal control individuals was done using the above said primers in a pooled reaction. PCR was carried out in a total volume of 5 μl containing 25 ng of genomic DNA, 1.0 pmol each of 6-FAM-labeled reverse primers and non-labeled forward primers, 1.5 Mm MgCl2, 0.25 mM of each dNTP, 0.03 U/μl of Taq DNA polymerase (Bangalore Genie, India) and the buffer recommended by the supplier. After PCR, 1 μl of the PCR product was loaded with and internal size standard (PET labeled) on ABI Prism 3100 Genetic Analyzer (Applied Biosystems). Fragment lengths were determined using the (Genotyper 3.7, Applied Biosystems). PCR was set up with the following conditions: Denaturation at 94 degree C. for 5 minutes, Thirty five cycles of denaturation at 94 degree C. for 30 seconds, annealing at 65 degree C. for 30 seconds, extension at 72 degree C. for 30 seconds; followed by a 10 minutes 72 degree C. segment extension period. To demonstrate the association of the D2S2311 repeat locus with atopic disorders such as asthma, CLUMP software analysis was used. CLUMP is a program designed to assess the significance of the departure of observed values in a contingency table from the expected values conditional on the marginal totals. The significance is assessed using a Monte Carlo approach, by performing repeated simulations to generate tables having the same marginal totals as the one under consideration, and counting the number of times that a chi-squared value associated with the real table is achieved by the randomly simulated data. This means that the significance levels assigned should be unbiased (with accuracy dependent on the number of simulations performed) and that no special account needs to be taken of continuity corrections or small expected values. This analysis showed significant differences between the allele count distribution of patient and control groups (χ2=43.441128, DF=9, p value<0.0001). We observed a significantly different pattern of distribution of the alleles between the two groups; alleles 402 and 404 were over-represented in the patient group whereas alleles 400 and 406 were the major alleles in the control group. Allele 402 was found to be a risk allele with Odds Ratio 2.289, 95% C.I. [1.5443-3.3929] allele 400 was found to be a protective allele with Odds Ratio 0.575, 95% C.I. [0.4098-0.8068] as calculated by 2×2 table (http://home.clara.net/sisa/twoby2.htm) (FIG. 3). Similar results were obtained in family based study (p value=0.0007) (Table 3a).

### EXAMPLE 2

Association of +92031 A/T Polymorphism with Atopic Disorders Such as Asthma:

The 1036 bp DNA stretch of SEQ ID No. 4 of INPP4A gene having the +92031 A/T polymorphism was PCR amplified using novel primers of SEQ ID No. 13 and 14. The genotyping was done using SNaPshot. ddNTP Primer Extension Kit (Applied Biosystems, Foster City, USA). SnaPshot PCR was carried out using 50 ng purified PCR template, 1 pmol primer with SEQ ID 19 and ABI ready reaction mix and 1× dilution buffer (as supplied by the manufacturer). PCR was set up with the following conditions: 96° C. for 10 seconds, 58° C. for 5 seconds and 60° C. for 30 seconds for a total of 30 cycles. To clean up the primer extension reaction, 1 U of calf intestinal phosphatase (CIP) diluted in 10× NEB3 (New England Biolabs), was added to the reaction mixture and the mixture was incubated at 37° C. for 1 hour, followed by an incubation for 15 minutes at 72° C. for enzyme inactivation. These samples were subsequently electrophoresed using the ABI Prism 3100 Genetic Analyzer as per the manufacturer's instructions. The results were analyzed using the program ABI Prism GeneScan™ and Genotyper™ (Applied Biosystems, Foster City, USA). To demonstrate the association of +92031 A/T polymorphism, Armitage trend test was performed. The pattern of distribution of the three genotypes, AA, AT and TT was significantly different in the two groups studied (χ2=7.05, p=0.008). The A allele was predominant in the cases as compared to the control group {OR=1.542, 95% C.I. (1.121-2.120), p=0.0075} (Table 1, FIG. 4). Similarly, the A allele was found to be over transmitted to affected offsprings in family based study using TDT-sTDT program (χ2=9, p=0.0027) (Table 3c).

### EXAMPLE 3

Association of +110832 A/G Polymorphism with Atopic Disorders Such as Asthma:

The 961 bp DNA stretch of SEQ ID No. 5 of INPP4A gene having the +110832 A/G polymorphism was PCR amplified using novel primers of SEQ ID No. 15 and 16. The genotyping was done using SNaPshot. ddNTP Primer Extension Kit (Applied Biosystems, Foster City, USA). SnaPshot PCR was carried out using 50 ng purified PCR template, 1 pmol primer with SEQ ID 22 and ABI ready reaction mix and 1× dilution buffer (as supplied by the manufacturer). PCR was set up with the following conditions: 96° C. for 10 seconds, 58° C. for 5 seconds and 60° C. for 30 seconds for a total of 30 cycles. To clean up the primer extension reaction, 1 U of calf intestinal phosphatase (CIP) diluted in 10× NEB3 (New England Biolabs), was added to the reaction mixture and the mixture was incubated at 37° C. for 1 hour, followed by an incubation for 15 minutes at 72° C. for enzyme inactivation. These samples were subsequently electrophoresed using the ABI Prism 3100 Genetic Analyzer as per the manufacturer's instructions. The results were analyzed using the program ABI Prism GeneScan™ and Genotyper™ (Applied Biosystems, Foster City, USA). To demonstrate the association of +110832 A/G polymorphism, Armitage trend test was performed in case-control study. The risk allele A showed a marginal association with the disease phenotype {Odds_{— ratio=}1.324, 95% C.I. (0.964-1.819), χ2=3.01, p=0.08) (Table 1). The A allele was found to be over transmitted to affected offsprings in family based study using TDT-sTDT program (χ2=11.5, p=0.0007) (Table 3e).

### EXAMPLE 4

Association of INPP4A M1_ S1_ M3 _S4 Locus Haplotype with Atopic Disorders Such as Asthma:

To demonstrate haplotypic association PHASE program was used to generate haplotypes for the patient and control groups. The program PHASE implements a new statistical method for reconstructing haplotypes from population genotype data. Experiments with the software on both real and simulated data indicate that it can provide an improvement on the EM algorithm for reconstructing haplotypes. It allows for missing genotype data and also can handle more than one locus irrespective of the polymorphism, for example SNPs and repeats can be analyzed simultaneously. Based on the output from the software the probability values of the haplotypes are also predicted and can be utilized to differentiate more confident haplotypes. The PHASE software is suitable for genetic distances of 100 cM or less. Similarly, family based haplotypic analysis was performed using TRANSMIT program. TRANSMIT tests for association between genetic marker and disease by examining the transmission of markers from parents to affected offspring. The tests are based on a score vector which is averaged over all possible configurations of parental haplotypes and transmissions consistent with the observed data.

In case-control study, the haplotypes whose expected frequency was larger than 0.025, in either of the two groups are shown in Table 2 (FIG. 6). The odds in favor of patients rather than controls having 402_A_{—}154_A haplotype was 3.68 with 95% CI: 2.2977, 5.916. The corresponding likelihood ratio χ2 tests showed p-value less than 10-5. TRANSMIT also showed similar results in family based association study (χ2=4.2714, DF=1, p value=0.038). Thus the 4-locus haplotype, comprising loci M1_ S1_ M3 _S4, 402_A_{—}154_A was strongly associated with asthma. On the other hand, the odds in favor of patients rather than controls having haplotype 400_T_{—}152_G and 400_A_{—}154_A were 0.16, 95% CI: 0.46-1.09 and 0.12, 95% CI: 0.33-0.82 respectively. The haplotype 400_T_{—}152_G was also found to be negatively associated with occurrence of atopic asthma in family based study. (χ²=8.065, DF=1, p value=0.0045). Thus, haplotypes 402_A_{—}154_A and 400_T_{—}152_G were identified to be major risk and protective haplotypes respectively.

### EXAMPLE 5

PEST Analysis of +110832 A/G Polymorphism:

To demonstrate the importance of Threonine to Alanine substitution at amino acid 604 in the α3 splice variant of INPP4A gene. The extended exon contains potential PEST sequence from amino acid 584-607 with PESTfind score of +7.49 (www.at.embnt.org/embnet/tools/bio/pestfind/). The +110832 A/G (rs2278206) polymorphism causes a threonine to alanine substitution at position 604 in protein sequence resulting in a poor PEST sequence (PESTfind score +4.95). Thus, Threonine to Alanine substitution at this particular locus can make the INPP4 enzyme resistant to calpain proteases (FIG. 7A).

### EXAMPLE 6

Functional Validation of +110832 A/G Polymorphism:

To substantiate this hypothesis experimentally, platelets from normal healthy individuals of different genotypes at A+110832G (S4) were stimulated with ionomycin and the degradation of INPP4A was checked by western blot analysis. Protein from platelets of individuals with AA genotype was found to be more susceptible to degradation as seen by nearly 60% degradation within 10 min of stimulation whereas the protein from individuals with GG genotype was less susceptible (nearly 31% degradation). Also, the basal levels of INPP4A protein from individuals with AA genotypes was found to be lower (data not shown), suggesting poor stability in vivo as well (FIG. 7b, 7c).

### EXAMPLE 7

Novel Splice Variants of INPP4A Gene:

To demonstrate the presence of novel splice variants of INPP4A gene expressed in blood of atopic asthmatic patients. RT PCR was carried out from the RNA of 12 atopic asthmatics and 4 normal healthy individuals. RNA was isolated from the total leukocytes using EZ-RNA isolation kit, following manufacturer's directions (Biological Industries). cDNA was made from 10 μg of total RNA using cDNA Archive kit from Applied Biosystems. PCR was carried out in a total volume of 20 μl containing cDNA corresponding to 100 ng of starting RNA, 2.0 pmol each of forward and reverse primer (SEQ ID 25 and 26 respectively), 1.5 Mm MgCl₂, 0.25 mM of each dNTP, 0.03 U/μl of Taq DNA polymerase (Bangalore Genie, India) and the buffer recommended by the supplier. PCR conditions: Denaturation at 94 degree C. for 5 minutes, 30 cycles of denaturation at 94 degree C. for 45 seconds, annealing at 65 degree C. for 45 seconds, extension at 72 degree C. for 45 seconds; followed by a 10 minutes 72 degree C. segment extension period. To demonstrate the presence of novel splice variants of INPP4A gene expressed in blood of topic asthmatic patients. RT PCR was carried out from the RNA of 12 atopic asthmatics and 4 normal healthy individuals. Two additional splice variants apart from the reported a 3 were identified in four atopic asthmatic individuals, whereas no splice variant was observed in the normal healthy individuals (FIG. 8).

### EXAMPLE 8

Allergen Induced Expression of INPP4A Protein Profile:

The BALB/c mice were sensitized by intra-peritoneal injections of 20 μg chicken egg ovalbumin (OVA) (grade V≧98% pure, Sigma Chemical Co., St. Louis, Mo., USA) in 0.2 ml saline adsorbed on 2 mg alum (sensitized) or alum alone (control) on days 0, 7 and 14. Mice were then challenged with aerosolized 3% ovalbumin (sensitized) or aerosolized saline (control) 30 minutes per day for 10 consecutive days (from day 22 to day 32). Responsiveness to methacholine (Sigma-Aldrich) was assessed in conscious, unrestrained mice by barometric plethysmography, using protocol, apparatus and software supplied by BUXCO (Troy, N.Y., USA). Mice were sacrificed 16 hrs after the last challenge and lung tissue were taken for immunohistochemistry. Immunohistochemistry of mouse lung section was done using commercial goat polyclonal antibody sc-12315 (Santa Cruz Biotechnology, Inc.; CA, USA), raised against a peptide near the amino terminus of INPP4A of human and mouse origin following standard protocol with slight modifications (Ather M H et al, 2004). Goat gamma globulin was used as isotype control (Jackson Immunoresearch Laboratories, Inc.; PA, USA).

The INPP4A protein was seen to be expressed at lower levels in ovalbumin induced mouse model of asthma than in the saline treated controls (FIG. 9).

| | | | |
|---|---|---|---|
| SNP ID | Genotype | Patients (N = 192) | Controls (N = 272) |

### EXAMPLE 9

Restoration of INPP4A Protein by Treatment with Anti-Inflammatory Agent such as Steroid:

The BALB/c mice were sensitized by intra-peritoneal injections of 20 μg chicken egg ovalbumin (OVA) (grade V≧98% pure, Sigma Chemical Co., St. Louis, Mo, USA) in 0.2 ml saline adsorbed on 2 mg alum on days 0, 7 and 14. Mice were then challenged with aerosolized 3% ovalbumin 30 minutes per day for 10 consecutive days (from day 22 to day 32). Treatment with 1 mg/kg dexamethasone was given starting from the day of last sensitization to last challenge (day 14-32). Bronchial hyperresponsiveness was measured as described previously using BUXCO plethysmograph (Troy, N.Y., USA). Mice were sacrificed 16 hrs after the last OVA challenge and lungs were subjected to immunohistochemistry. Immunohistochemistry of mouse lung section was done using commercial goat polyclonal antibody sc-12315 (Santa Cruz Biotechnology, Inc.; CA, USA), raised against a peptide near the amino terminus of INPP4A of human and mouse origin following standard protocol with slight modifications (Ather M H et al, 2004). Goat gamma globulin was used as isotype control (Jackson Immunoresearch Laboratories, Inc.; PA, USA). The levels of INPP4A protein were found to be restored to normal levels in case of steroid treated ovalbumin induced mouse model of asthma (FIG. 9).

Alternative embodiments of the invention can be envisaged by those skilled in the art from the information contained herein. All such alternative embodiments are intended to lie within the scope of this application.

All the features disclosed in this specification (including any accompanying claims, abstract and drawing), and/or all of the steps or any method or process so disclosed, may be combined in any combination, except combination where at least some of such features and/or steps are mutually exclusive.

TABLE 1
Frequency (%) of SNPs in patients and controls.

| | | | |
|---|---|---|---|
| +92031 A/T | TT | 3.19 | 7.78 |
| (rs3769712) | AT | 32.45 | 38.15 |
| | AA | 64.36 | 54.07 |
| +92344 C/T | TT | 0.54 | 1.13 |
| (rs3769710) | CT | 13.98 | 19.17 |
| | CC | 85.48 | 79.7 |
| +92817 C/T | TT | 3.51 | 7.48 |
| (rs2278208) | CT | 33.92 | 37.4 |
| | CC | 62.57 | 55.12 |
| +110832 A/G | GG | 3.3 | 7.58 |
| (rs2278206) | AG | 34.62 | 36.74 |
| | AA | 62.09 | 55.68 |
| +131237 C/T | TT | 7.61 | 6.67 |
| (rs10201079) | CT | 40.76 | 32.22 |
| | CC | 51.63 | 61.11 |

Each feature disclosed in this specification (including any accompanying claims, abstract and drawings), may be replaced by alternative features serving the same or equivalent or similar purpose, unless expressly stated otherwise. Thus, unless expressly slated otherwise, each feature disclosed is one example only of a generic series of equivalent or similar features.

The invention is not restricted to the details of the embodiments. This invention extends to any novel one, or any novel combination, or the features disclosed in this specification (including any accompanying claims, abstract and drawings), or to any novel one, or any novel combination, of the steps of any method or process so disclosed.

TABLE 2
Frequency (%) of haplotypes constructed using D2S311/rs3769712/CA
repeat/rs2278206 markers in patients and controls estimated by PHASE.
Haplotypes with relative frequencies >0.025 (2.5% of sample size)
in either of the groups have been depicted below.

| S. NO. | HAPLOTYPE | PATIENTS | CONTROLS |
|---|---|---|---|
| 1. | 398_A_154_A | 2.86 | 0.91 |
| 2. | 400_T_152_G | 9.11 | 12.32 |
| 3. | 400_A_154_A | 7.29 | 13.05 |
| 4. | 402_T_152_G | 1.04 | 3.49 |
| 5. | 402_A_154_A | 16.14 | 4.96 |
| 6. | 404_T_152_G | 6.25 | 5.51 |
| 7. | 404_A_154_A | 45.57 | 40.26 |
| 8. | 406_A_154_A | 2.60 | 6.62 |

TABLE 3(a)
D2S2311/M1 (p = 0.0007)

| Allele | Transmitted | Non-transmitted | Chi-Sq |
|---|---|---|---|
| 386 | 4 | 0 | 4 |
| 396 | 3 | 5 | 0.5 |
| 398 | 9 | 11 | 0.2 |
| 400 | 27 | 58 | 11.306 |
| 402 | 36 | 22 | 3.379 |
| 404 | 72 | 50 | 3.967 |
| 406 | 4 | 9 | 1.923 |
| 408 | 5 | 5 | 0 |

TABLE 3(b)
D2S2187/M2 (p = 0.19)

| Allele | Transmitted | Non-transmitted | Chi-Sq |
|---|---|---|---|
| 224 | 2 | 1 | 0.333 |
| 226 | 1 | 1 | 0 |
| 228 | 6 | 5 | 0.091 |
| 230 | 13 | 21 | 1.882 |
| 232 | 34 | 41 | 0.653 |
| 234 | 84 | 62 | 3.315 |
| 236 | 39 | 44 | 0.301 |
| 238 | 15 | 24 | 2.077 |
| 240 | 4 | 2 | 0.667 |
| 244 | 3 | 0 | 3 |

TABLE 3(c)
rs3769712/SS1 (p = 0.0027)

| Allele | Transmitted | Non-transmitted | Chi-Sq |
|---|---|---|---|
| A | 65 | 35 | 9 |
| T | 35 | 65 | 9 |

TABLE 3(d)
SS2 rs3769710 (p = 0.058)

| Allele | Transmitted | Non-transmitted | Chi-Sq |
|---|---|---|---|
| C | 30 | 17 | 3.596 |
| T | 17 | 30 | 3.596 |

TABLE 3(e)
rs2278208/SS3 (p = 0.063)

| Allele | Transmitted | Non-transmitted | Chi-Sq |
|---|---|---|---|
| C | 56 | 38 | 3.447 |
| T | 38 | 56 | 3.447 |

TABLE 3(f)
CA Repeat (p = 0.008)

| Allele | Transmitted | Non-transmitted | Chi-Sq |
|---|---|---|---|
| 152 | 38 | 65 | 7.078 |
| 154 | 65 | 38 | 7.078 |

TABLE 3(g)
rs2278206/SS4 (p = 0.0007)

| Allele | Transmitted | Non-transmitted | Chi-Sq |
|---|---|---|---|
| A | 78 | 41 | 11.504 |
| G | 41 | 78 | 11.504 |

TABLE 3(h)
rs10201079/SS5 (p = 0.03)

| Allele | Transmitted | Non-transmitted | Chi-Sq |
|---|---|---|---|
| C | 34 | 54 | 4.545 |
| T | 54 | 34 | 4.545 |

### REFERENCES

1. Abney M, Ober C, McPeek M S (2002) Quantitative-trait homozygosity and association mapping and empirical genomewide significance in large, complex pedigrees: fasting serum-insulin level in the Hutterites. Am J Hum Genet 70:920-34.

2. Alam R et al (1994) Transforming growth factor beta abrogates the effects of hematopoietins on eosinophils and induces their apoptosis. J Exp Med 179: 1041-1045.

3. Altshuler D, Kruglyak L, Lander E (1998) Genetic polymorphisms and disease. N Engl J Med 338:1626.

4. Ather M H, Abbas F, Faruqui N, Israr M, Pervez S. Expression of pS2 in prostate cancer correlates with grade and Chromogranin A expression but not with stage. BMC Urol. 2004; 4(1):14.

5. Aubert J D, Dalal B I, Bai T R et al (1994) Transforming growth factor β1 gene expression in human airways. Thorax 49:225-232.

6. Babron M C, Selinger-Leneman H, Dizier M H, Clerget-Darpoux F (2001) Homogeneity of asthma genome scan results. Genet Epidemiol 21 Suppl 1:S44-8.

7. Barringer K J, Orgel L, Wahl G, Gingeras T R (1990) Blunt-end and single-strand ligations by Escherichia coli ligase: influence on an in vitro amplification scheme. Gene 89:117-22.

8. Baron M (2001) The search for complex disease genes: fault by linkage or fault by association? Mol Psychiatry 6:143-9.

9. Barnes P J (2001) Th2 cytokines and asthma: an introduction. Respir Res 2:64-5.

10. Barnes K C (1999) Gene-environment and gene-gene interaction studies in the molecular genetic analysis of asthma and atopy. Clin Exp Allergy 29 Suppl 4:47-51.

11. Barnes K C, Neely J D, Duffy D L, Freidhoff L R, Breazeale D R, Schou C, Naidu R P, et al (1996) Linkage of asthma and total serum IgE concentration to markers on chromosome 12q: evidence from Afro-Caribbean and Caucasian populations. Genomics 37:41-50.

12. Barnes P J. New aspects of asthma. J Intern Med 1992; 231:453-61

13. Ben-Asouli Y, Banai Y, Pel-Or Y, Shir A, Kaempfer R (2002) Human interferon-gamma mRNA autoregulates its translation through a pseudoknot that activates the interferon-inducible protein kinase PKR. Cell 108:221-32.

14.Bleecker E R, Postma D S, Meyers D A (1997) Evidence for multiple genetic susceptibility loci for asthma. Am J Respir Crit Care Med 156:S113-6.

15. Blumenthal M N, Amos D B (1987) Genetic and immunologic basis of atopic responses. Chest 91:176S-184S.

16. Blumenthal M N, Langefeld C D (2004) A genome-wide search for allergic response (atopy) genes in three ethnic groups: Collaborative Study on the Genetics of asthma. Hum Genet 114(2):157-164.

17. Blumenthal M N, Ober C (2004) Genome scan for loci linked to mite sensitivity: the Collaborative Study on the Genetics of Asthma (CSGA). Genes Immun 5(3):226-231.

18. Bodmer W F (1987) The human genome sequence and the analysis of multifactorial traits. Ciba Found Symp 130:215-28.

19. Breslow J L (1988) Apolipoprotein genetic variation and human disease. Physiol Rev 68:85-132.

20. Caraballo L R, Hernandez M (1990) HLA haplotype segregation in families with allergic asthma. Tissue Antigens 35:182-6.

21. Cargill M, Altshuler D, Ireland J, Sklar P, Ardlie K, Patil N, Shaw N, et al (1999) Characterization of single-nucleotide polymorphisms in coding regions of human genes. Nat Genet 22:231-8.

22. Carriere K C, Kochar S C (2000): Comparing sub-survival functions in a competing risks model.Lifetime Data Anal.: 6(1):85-97.

23. Chang C, Bowman J L, DeJohn A W, Lander E S, Meyerowitz E M (1988) Restriction fragment length polymorphism linkage map for Arabidopsis thaliana. Proc Natl Acad Sci USA 85:6856-60.

24. Christie L, Hine R J, Parker J G, Burks W (2002). Food allergies in children affect nutrient intake and growth. J Am Diet Assoc. 102 (11):1648-51.

25. Cohen D, Cohen O, Marcadet A, Massart C, Lathrop M, Deschamps I, Hors J, et al (1984) Class II HLA-DC beta-chain DNA restriction fragments differentiate among HLA-DR2 individuals in insulin-dependent diabetes and multiple sclerosis. Proc Natl Acad Sci USA 81:1774-8.

26. Cookson W O, Young R P, Sandford A J, Moffatt M F, Shirakawa T, Sharp P A, Faux J A, et al (1992) Maternal inheritance of atopic IgE responsiveness on chromosome 11q. Lancet 340:381-4.

27. Cookson W (1999) The alliance of genes and environment in asthma and allergy. Nature 402:B5-11.

28. Duffy D L (1997) Genetic epidemiology of asthma. Epidemiol Rev 19:129-43.

29. Elias J A, Zheng T, Lee C G, Homer R J, Chen Q, Ma B, Blackburn M, Zhu Z. Transgenic Modeling of Interleukin-13 in the Lung. Chest. March 2003;123(3 Suppl):339S-45S.

30. Erlich H A (eds), Freeman Press, N.Y., N.Y., 1992); PCR Protocols: A Guide to Methods and Applications (eds. Innis, et al., Academic Press, San Diego, Calif., 1990.

31. Friedhoff P, Hahn M, Wolfes H, Pingoud A (1993) Quantitative polymerase chain reaction with oligodeoxynucleotide ligation assay/enzyme-linked immunosorbent assay detection. Anal Biochem 215:9-16.

32. Fujii D, Brissenden J. E, Derynck R, Francke U (1986) Transforming growth factor beta gene maps to human chromosome 19 long arm and to mouse chromosome 7. Somat Cell Mol Genet 12:281-288

33. de Gouyon B, Melanitou E, Richard M F, Requarth M, Hahn I H, Guenet J L, Demenais F, et al (1993) Genetic analysis of diabetes and insulitis in an interspecific cross of the nonobese diabetic mouse with Mus spretus. Proc Natl Acad Sci USA 90:1877-81.

34. Grainger D J, Heathcote K, Chiano M, Sneider H, Kemp P R et al Genetic control of the circulating concentration of transforming growth factor β1. Hum Mol Gen 8:93-97

35. Hacia J G, Fan J B, Ryder O, Jin L, Edgemon K, Ghandour G, Mayer R A, et al (1999) Determination of ancestral alleles for human single-nucleotide polymorphisms using high-density oligonucleotide arrays. Nat Genet 22:164-7.

36. Herd C M, Page C P. Pulmonary immune cells in health and disease: platelets. Eur Respir J 1994; 7:1145-60

37. Hefferon W T, Groman D J, Yurk E C, Garry R C (2004) A variable dinucleotide repeat in the CFTR gene contributes to phenotype diversity by forming RNA secondary structures that alter splicing. PNAS 101(10):3504-3509.

38. Heinzmann A, Grotherr P, Jerkic S P, Lichtenberg A, Braun S, Kruse S, Forster J, et al (2000) Studies on linkage and association of atopy with the chromosomal region 12q13-24. Clin Exp Allergy 30:1555-61.

39. Hirschhorn J N, Sklar P, Lindblad-Toh K, Lim Y M, Ruiz-Gutierrez M, Bolk S, Langhorst B, et al (2000) SBE-TAGS: an array-based method for efficient single-nucleotide polymorphism genotyping. Proc Natl Acad Sci USA 97:12164-9.

40. James E. Gern, Robert F. Lemanske, Jr. and William W. Busse (1999) Early life origins of asthma. J Clin Invest, 104:7, 837-843

41. Julier C, de Gouyon B, Georges M, Guenet J L, Nakamura Y, Avner P, Lathrop G M (1990) Minisatellite linkage maps in the mouse by cross-hybridization with human probes containing tandem repeats. Proc Natl Acad Sci USA 87:4585-9.

42. Kameyoshi Y, Do\ddot{} rchner A, Mallet A I, et al. (1992) RANTES released by thrombin-stimulated platelets is a potent attractant for human eosinophils. J Exp Med; 176:587-92

43. Kameyoshi Y, Schro\ddot{} der J M, Christopher E, et al. (1994) Identification of the cytokine RANTES released from platelets as an eosinophil chemotactic factor. Int Arch Allergy Immunol; 104(suppl 1):49-51

44. Klinger M H, Wilhelm D, Bubel S, et al. (1995) Immunocytochemical localization of the chemokines RANTES and MIP-1 alpha within human platelets and their release during storage. Int Arch Allergy Immunol; 107:541-46

45. Kreutz R, Hubner N, James M R, Bihoreau M T, Gauguier D, Lathrop G M, Ganten D, et al (1995) Dissection of a quantitative trait locus for genetic hypertension on rat chromosome 10. Proc Natl Acad Sci USA 92:8778-82.

46. Kwoh D Y, Davis G R, Whitfield K M, Chappelle H L, DiMichele L J, Gingeras T R (1989) Transcription-based amplification system and detection of amplified human immunodeficiency virus type 1 with a bead-based sandwich hybridization format. Proc Natl Acad Sci USA 86:1173-7.

47. Lander E S, Botstein D (1986) Strategies for studying heterogeneous genetic traits in humans by using a linkage map of restriction fragment length polymorphisms. Proc Natl Acad Sci USA 83:7353-7.

48. Lander E S (1993) Finding similarities and differences among genomes. Nat Genet 4:5-6.

49. Lathrop G M, Lalouel J M, Julier C, Ott J (1984) Strategies for multilocus linkage analysis in humans. Proc Natl Acad Sci USA 81:3443-6.

50. Levitt R C (1994) Molecular genetic methods for mapping disease genes. Am J Respir Crit Care Med 150:S94-9.

51. Longo D R, Johnson J C, Kruse R L, Brownson R C, Hewett J E (2001): A prospective investigation of the impact of smoking bans on tobacco cessation and relapse. Tob Control, Sep;10(3):267-72.

52. Mattila P, Korpela J, Tenkanen T, Pitkanen K (1991) Fidelity of DNA synthesis by the Thermococcus litoralis DNA polymerase—an extremely heat stable enzyme with proofreading activity. Nucleic Acids Res 19:4967-73.

53. Mokdad-Gargouri R, Belhadj K, Gargouri A (2001) Translational control of human p53 expression in yeast mediated by 5′-UTR-ORF structural interaction. Nucleic Acids Res 29:1222-7.

54. Mukawa A, Kamitsuma Y, Tsunekawa S, Tanaka N (1989): Report on a long-term trial of CYBEST Model 2 for prescreening for squamous cell carcinoma of the uterine cervix Anal Cell Pathol. Aug; 1 (4):225-33.

55. Nagarkatti R, B Rao C, Rishi J P, Chetiwal R, Shandilya V, Vijayan V, Kumar R, Pemde H K, Sharma S K, Sharma S, Singh A B, Gangal S V, Ghosh B (2002) Association of IFNG gene polymorphism with asthma in the Indian population. JACI September 2002;110(3):410-2.

56. Norris F A, Atkins R C, Majerus P W. (1997) Inositol polyphosphate 4-phosphatase is inactivated by calpain-mediated proteolysis in stimulated human platelets. J Biol Chem. Apr 25;272(17):10987-9

57. Ober C, Tsalenko A, Parry R, Cox N J (2000) A second-generation genomewide screen for asthma-susceptibility alleles in a founder population. Am J Hum Genet 67:1154-62.

58. Oberle I, Heilig R, Moisan J P, Kloepfer C, Mattei G M, Mattei J F, Boue J, et al (1986) Genetic analysis of the fragile-X mental retardation syndrome with two flanking polymorphic DNA markers. Proc Natl Acad Sci USA 83:1016-20.

59. Orita M, Iwahana H, Kanazawa H, Hayashi K, Sekiya T (1989) Detection of polymorphisms of human DNA by gel electrophoresis as single-strand conformation polymorphisms. Proc Natl Acad Sci USA 86:2766-70.

60. Paul W E (1997) Interleukin 4: signaling mechanisms and control of T cell differentiation. Ciba Found Symp 204:208-16; discussion 216-9.

61. Piacquadio D J, Chen D M, Farber H F, Fowler J F Jr, Glazer S D, Goodman J J, Hruza L L, Jeffes E W, Ling M R, Phillips T J, Rallis T M, Scher R K, Taylor C R, Weinstein G D (2004): Photodynamic therapy with aminolevulinic acid topical solution and visible blue light in the treatment of multiple actinic keratoses of the face and scalp: investigator-blinded, phase 3, multicenter trials. Arch Dermatol.; 140(1): 41-6.

62. Reich D E, Cargill M, Bolk S, Ireland J, Sabeti P C, Richter D J, Lavery T, et al (2001) Linkage disequilibrium in the human genome. Nature 411:199-204.

63. Rothenburg S, Koch-Nolte F, Rich A, Haag F (2001) A polymorphic dinucleotide repeat in the rat nucleolin gene forms Z-DNA and inhibits promoter activity. Proc Natl Acad Sci USA 98:8985-90.

64. Sachidanandam R, Weissman D, Schmidt S C, Kakol J M, Stein L D, Marth G, Sherry S, et al (2001) A map of human genome sequence variation containing 1.42 million single nucleotide polymorphisms. Nature 409:928-33.

65. Sambrook et al., Molecular Cloning, A Laboratory Manual, 2nd Ed., CSHP, New York 1989.

66. Saiki R K, Bugawan T L, Horn G T, Mullis K B, Erlich H A (1986) Analysis of enzymatically amplified beta-globin and HLA-DQ alpha DNA with allele-specific oligonucleotide probes. Nature 324:163-6.

67.

68. Sham, P. C., and Curtis, D. (1995). “Monte Carlo tests for associations between disease and alleles at highly polymorphic loci.” Annals of Human Genetics, 59, 97-105.

69. Shearn C T, Walker J, Norris F A. (2001) Identification of a novel spliceoform of inositol polyphosphate 4-phosphatase type Ialpha expressed in human platelets: structure of human inositol polyphosphate 4-phosphatase type Igene. Biochem Biophys Res Commun. Aug 10;286 (1):119-25.

70. Simon C. Pitchford (2004) Platelets are necessary for airway wall remodeling in a murine model of chronic allergic inflammation. Blood:103, 2

71. Sorensen G, Emmons K, Stoddard A M, Linnan L, Avrunin J (2002): Do social influences contribute to occupational differences in quitting smoking and attitudes toward quitting? Am J Health Promot. Jan-Feb;16(3):135-41.

72. Stephens M, Donnelly P (2003): A comparison of bayesian methods for haplotype reconstruction from population genotype data. Am J Hum Genet. Nov 73:1162-9.

73. Tay A H, Tan E C, Chew F T, Goh D L, Shek L P, Lee B W (1999) Ethnic differences in genetic susceptibility to atopy and asthma. Asian Pac J Allergy Immunol 17:239-42.

74. Thomas N S, Wilkinson J, Holgate S T (1997) The candidate region approach to the genetics of asthma and allergy. Am J Respir Crit Care Med 156:S144-51.

75. Vyas P, Norris F A, Joseph R, Majerus P W, Orkin S H. (2000) Inositol polyphosphate 4-phosphatase type I regulates cell growth downstream of transcription factor GATA-1. Proc Natl Acad Sci USA. Dec 5;97 (25):13696-701

76. Xu J, Wiesch D G, Meyers D A (1998) Genetics of complex human diseases: genome screening, association studies and fine mapping. Clin Exp Allergy 28 Suppl 5:1-5; discussion 26-8.

77. Xu J, Meyers D A, Ober C, Blumenthal M N, Mellen B, Barnes K C, King R A, et al (2001) Genomewide screen and identification of gene-gene interactions for asthma-susceptibility loci in three U.S. populations: collaborative study on the genetics of asthma. Am J Hum Genet 68:1437-46.

1. Genetic variants of human Inositol polyphosphate 4-phosphatase (INPP4A) of h gene and the splice variants thereof useful for the prediction of susceptibility of an individual to immunological disorders, particularly asthma.

2. Genetic variants as claimed in claim 1, comprising 1 to 401 contiguous nucleotides containing one or more groups of CA dinucleotides at positions from 31 to 73 on M1 locus having SEQ ID No.1.

3. Genetic variants as claimed in claim 2, wherein the said variant is pharmacogenetic marker for predicting and detecting humans susceptible to asthma.

4. Genetic variant as claimed in claim 2, wherein the percentage frequency for allele 400 of the D2S2311 dinucleotide repeat polymorphism at M1 locus is 17.05% in patients.

5. Genetic variant as claimed in claim 2, wherein the D2S2311 dinucleotide repeat is on 400 allele of M1 locus and is negatively associated with asthma with odds ratio of 0.575, 95% C.I. [0.4098-0.8068] indicating low risk.

6. Genetic variant as claimed in claim 2, wherein the allele 402 of the gene variant of D2S2311 dinucleotide repeat polymorphism at M1 locus has a percentage frequency of about 19.32% in patients.

7. Genetic variants as claimed in claim 2, wherein the gene variant of D2S2311 dinucleotide repeat is on 402 allele on M1 locus and is positively associated with asthma with odds ratio equal to 3.68 with 95% CI: 2.2977, 5.916, indicating high risk.

8. Genetic variant as claimed in claim 2, wherein the gene variant of D2S2311 dinucleotide repeat polymorphism at M1 locus is associated with susceptibility to asthma χ2=43.441128, DF=9, p value<0.0001.

9. Genetic variants as claimed in claim 1, comprising 1 to 1036 contiguous nucleotides containing +92031 A/T polymorphism at position 75 on S1 locus having SEQ ID No.4.

10. Genetic variants as claimed in claim 1, comprising 1 to 159 contiguous nucleotides containing one or more groups of CA dinucleotides at positions from 105 to 139 on M3 locus having SEQ ID No.3.

11. Genetic variants as claimed in claim 1, comprising 1 to 1707 contiguous nucleotides containing +110832 A/G non-synonymous SNP (Ala/Thr) at position 1221 on S4 locus having SEQ ID No.6.

12. Genetic variants as claimed in, claim 1, wherein the said variants are useful for predicting and detecting humans susceptible to the immunological disorders selected from group comprising of asthma, autoimmune disorders, inflammatory disorders, cancer, multiple sclerosis, fibrosis, tuberculosis, sarcoidosis, hypertension and related disorders, diabetes and related disorders, alcohol abuse, anxiety, COPD, cholecystectomy, degenerative joint disease, seizure disorders, arthritis.

13. Genetic variants as claimed in, claim 1, wherein the said variant is pharmacogenetic marker for predicting and detecting humans susceptible to the immunological disorders selected from group comprising of asthma, autoimmune disorders, inflammatory disorders, cancer, multiple sclerosis, fibrosis, tuberculosis, sarcoidosis, hypertension and related disorders, diabetes and related disorders, alcohol abuse, anxiety, COPD, cholecystectomy, degenerative joint disease, seizure disorders, arthritis.

14. Genetic variants as claimed in claim 1, wherein the said variant is useful for predicting and detecting humans susceptible to asthma.

15. Genetic variants as claimed in claim 1, wherein the subject is human.

16. Genetic variants as claimed in claim 1, wherein the said variant is a pharmacogenetic marker useful for predicting and detecting humans susceptible to asthma.

17. Genetic variants as claimed in claim 1, which in combination form haplotype variants of M1_S1_M3_S4 loci in INPP4A gene.

18. Novel four-locus haplotypes as claimed in claim 17, the said novel haplotypes comprising 396_T_{—}154_G, 398_A_{—}152_A, 400_T_{—}152_A, 400_A_{—}152_A, 406_T_{—}152_A, 406_A_{—}156_T, 412_A_{—}154_A, 400_T_{—}154_A, 402_T_{—}152_A, 404_A_{—}156_T, 410_A_{—}154_A, 404_A_{—}152_G, 406_A_{—}152_A, 404_A_{—}152_A, 406_T_{—}152_G, 396_A_{—}152_G, 400_A_{—}154_G, 402_T_{—}154_A, 402_A_{—}152_A, 404_T_{—}154_A, 400_T_{—}154_G, 396_T_{—}152_G, 404_T_{—}152_A, 398_A_{—}152_G, 386_A_{—}154_A, 402_T_{—}152_G, 398_T_{—}152_G, 404_A_{—}154_G, 408_A_{—}154_A, 406_A_{—}154_A, 398_A_{—}154_A, 404_T_{—}152_G, 400_A_{—}154_A, 400_T_{—}152_G, 402_A_{—}154_A, 404_A_{—}154_A associated with M1_S1_M3_S4 loci.

19. Novel gene variants as claimed in claim 17, wherein the percentage frequency of M1_S1_M3_S4 locus on haplotype 402_A_{—}154_A is 16.14% in patients.

20. Novel gene variants as claimed in claim 17, wherein the haplotype 402_A_{—}154_A is strongly associated with occurrence of asthma (case-control: odds ratio 3.68 with 95% confidence interval: 2.2977, 5.916, p value<0.0001;
family based study: χ2=4.2714, degree of freedom=1, p value=0.038) indicating high risk.

21. Novel gene variant as claimed in claim 17, wherein the haplotype 400_A_{—}154_A and 400_T_{—}152_G is negatively associated with occurrence of asthma and Haplotype 400_T_{—}152_G is negatively associated with occurrence of atopic asthma in family based study (χ2=8.065, degree of freedom=1, p value=0.0045) indicating low risk.

22. Gene variants as claimed in claim 1, wherein the gene variants at D2S2311 dinucleotide repeat polymorphism is associated with susceptibility to asthma with p value<0.0001.

23. Gene variants as claimed in claim 1, wherein the gene variant on D2S2311 dinucleotide repeat polymorphism allele 402 is a risk allele with OR-2.289, 95% C.I. [1.5443-3.3929].

24. Gene variants as claimed in claim 1, wherein the gene variant on D2S2311 dinucleotide repeat polymorphism allele 400 is a protective allele with Odds Ratio 0.575, 95% C.I. [0.4098-0.8068].

25. Gene variants as claimed in claim 1, wherein the gene variants at +92031 A/T at S1 locus is associated with susceptibility to asthma with p value=0.00791 in case control study and p value=0.0027 in family based study.

26. Gene variants as claimed in claim 1, wherein the gene variants +110832 A/G (rs2278206) causing Ala/Thr substitution is associated with susceptibility to asthma.

27. Gene variants as claimed in claim 1, resulting in splice variants such as the novel splice variant wherein exon 16 is deleted.

28. Gene variants as claimed in claim 1, resulting in differential expression profile of the INPP4A gene as observed in mouse model of asthma.

29. A method for detecting and predicting predisposition to immunological disorders by screening for INPP4A gene and splice variants and its expression in a subject and the said method comprises the steps of:
(i) isolating DNA from samples selected from group comprising of whole blood, semen, saliva, tears, urine, fecal material, sweat, buccal, skin or hair;
(ii) designing and synthesizing primers having SEQ ID Nos. 7-23;
(iii) amplifying the genomic DNA using primers of SEQ ID Nos. 7, 8, 9, 10, 11, 12, 13, 14, 15, 16, 17, 18 wherein primers of SEQ ID Nos 7, 9, 11, 13, 15, 17 are forward primers and primers of SEQ ID Nos 8, 10, 12, 14, 16, 18 are reverse primers;
(iv) isolating and identifying the DNA stretch of SEQ ID No.1 using novel primer combinations of SEQ ID Nos. 7 and 8;
(v) isolating and identifying the DNA stretch of SEQ ID No.2 using primer combinations of SEQ ID Nos. 9 and 10;
(vi) isolating and identifying the DNA stretch of SEQ ID No.3 using primer combinations of SEQ ID Nos. 11 and 12;
(vii) isolating and identifying the DNA stretch of SEQ ID No.4 using primer combinations of SEQ ID Nos. 13 and 14;
(viii) isolating and identifying the DNA stretch of SEQ ID No.5 using primer combinations of SEQ ID Nos. 15 and 16;
(ix) isolating and identifying the DNA stretch of SEQ ID No.6 using primer combinations of SEQ ID Nos. 17 and 18;
(x) sequencing the isolated and identified SEQ ID Nos. 4, 5 and 6 obtained in steps (vi, vii and viii);
(xi) validating and identifying the specific INPP4A gene variants computationally by comparison with the known wild type INPP4A gene sequences;
(xii) isolating RNA from samples selected from group comprising of whole blood; and
(xiii) isolating and identifying splice variants of SEQ ID 24 using primer combinations of SEQ ID Nos.25 and 26.

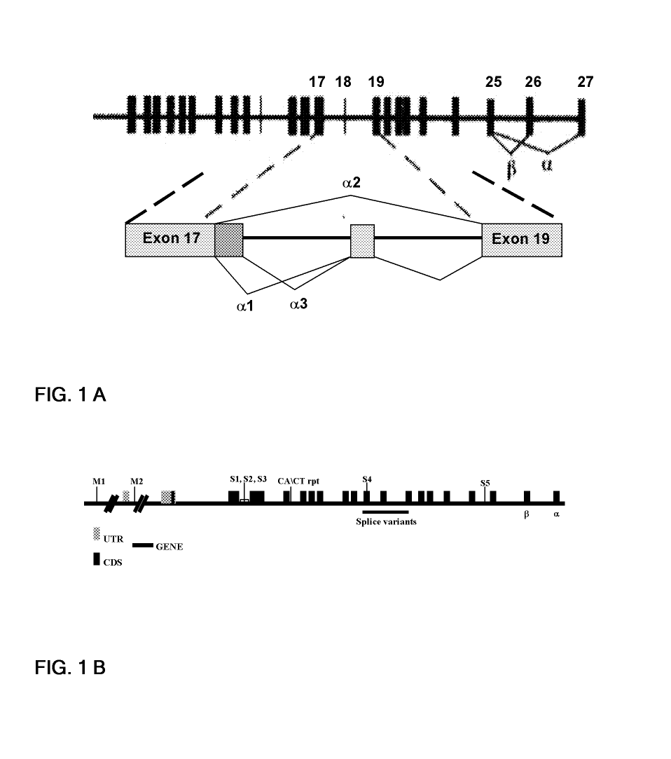

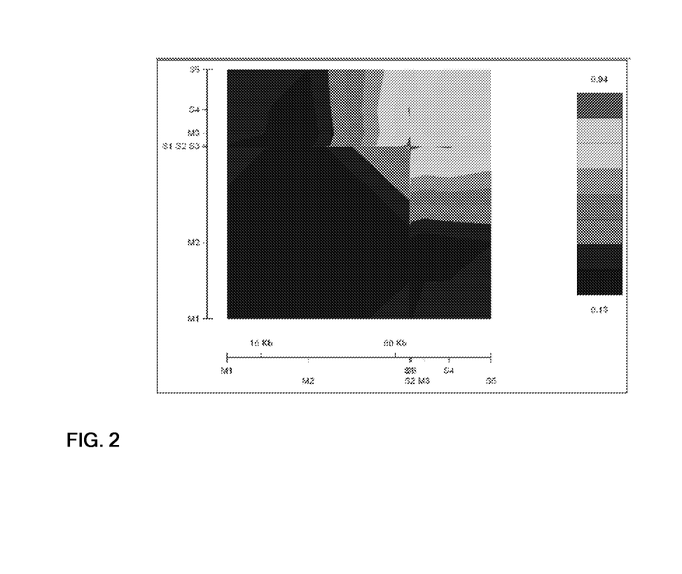

30. A method as claimed in claim 29, wherein SEQ ID NO:1 is associated with micro-satellite repeat 1 as is represented in FIG. 1, SEQ ID NO:2 is associated with micro-satellite repeat 2 as is represented in FIG. 1, SEQ ID NO:3 is associated with micro-satellite repeat 3 as is represented in FIG. 1, SEQ ID NO:4 is associated with SNP 1, SNP 2 and SNP 3 as represented in FIG. 1, SEQ ID NO:5 is associated with SNP 4 as represented in FIG. 1, SEQ ID NO:6 is associated with SNP 5 locus of the INPP4A gene as represented in FIG. 1.

31. A method as claimed in claim 29, wherein the subject is human.

32. A method as claimed in claim 29, wherein the said variants are useful for predicting and detecting humans susceptible to immunological disorders selected from group comprising of asthma, autoimmune disorders, inflammatory disorders, cancer, multiple sclerosis, fibrosis, tuberculosis, sarcoidosis, hypertension and related disorders, diabetes and related disorders, alcohol abuse, anxiety, COPD, cholecystectomy, degenerative joint disease, seizure disorders, arthritis.

33. A method as claimed in claim 29, wherein the said variants are useful for predicting and detecting humans susceptible to asthma.

34. A method as claimed in claim 29, wherein the said variants are pharmacogenetic markers for predicting and detecting humans susceptible to immunological disorders selected are from group comprising of asthma, autoimmune disorders, inflammatory disorders, cancer, multiple sclerosis, fibrosis, tuberculosis, sarcoidosis, hypertension and related disorders, diabetes and related disorders, alcohol abuse, anxiety, COPD, cholecystectomy, degenerative joint disease, seizure disorders, arthritis.

35. A method of preparing novel pharmacogenetic markers for detecting and predicting predisposition to immunological disorders of INPP4A gene in a subject, said method comprising of:
(i) isolating DNA from samples of whole blood, semen, saliva, tears, urine, fecal material, sweat, buccal, skin or hair,
(ii) designing and synthesizing primers having SEQ ID Nos. 7-23,
(iii) amplifying the genomic DNA using primers of SEQ ID Nos. 7, 8, 9, 10, 11, 12, 13, 14, 15, 16, 17, 18 wherein primers of SEQ ID Nos 7, 9, 11, 13, 15, 17 are forward primers and primers of SEQ ID Nos 8, 10, 12, 14, 16, 18 are reverse primers,
(iv) isolating and identifying the DNA stretch of SEQ ID No.1 using novel primer combinations of SEQ ID Nos.7 and 8,
(v) isolating and identifying the DNA stretch of SEQ ID No.2 using primer combinations of SEQ ID Nos.9 and 10,
(vi) isolating and identifying the DNA stretch of SEQ ID No.3 using primer combinations of SEQ ID Nos.11 and 12,
(vii) isolating and identifying the DNA stretch of SEQ ID No.4 using primer combinations of SEQ ID Nos. 13 and 14,
(viii) isolating and identifying the DNA stretch of SEQ ID No.5 using primer combinations of SEQ ID Nos.15 and 16,
(ix) isolating and identifying the DNA stretch of SEQ ID No.6 using primer combinations of SEQ ID Nos.17 and 18,
(x) sequencing the isolated and identified SEQ ID Nos. 4, 5 and 6 obtained in steps (vii, viii and ix),
(xi) validating and identifying the specific INPP4A gene variants computationally by comparison with the known wild type INPP4A gene sequences,
(xii) isolating RNA from samples selected from group comprising of whole blood, and
(xiii) isolating and identifying splice variants of SEQ ID 24 using primer combinations of SEQ ID Nos.25 and 26.

36. A method as claimed in claim 35, wherein SEQ ID NO:1 is associated with micro-satellite repeat 1 as is represented in FIG. 1, SEQ ID NO:2 is associated with micro-satellite repeat 2 as is represented in FIG. 1, SEQ ID NO:3 is associated with micro-satellite repeat 3 as is represented in FIG. 1, SEQ ID NO:4 is associated with SNP 1, SNP 2 and SNP 3 as represented in FIG. 1, SEQ ID NO:5 is associated with SNP 4 as represented in FIG. 1, SEQ ID NO:6 is associated with SNP 5 locus of the INPP4A gene as represented in FIG. 1.

37. A method as claimed in claim 35, wherein the subject is human.

38. A method as claimed in claim 35, wherein the said variants are useful for predicting and detecting humans susceptible to immunological disorders selected from group comprising of asthma, autoimmune disorders, inflammatory disorders, cancer, multiple sclerosis, fibrosis, tuberculosis, sarcoidosis, hypertension and related disorders, diabetes and related disorders, alcohol abuse, anxiety, COPD, cholecystectomy, degenerative joint disease, seizure disorders, arthritis.

39. Pharmacogenetic markers having SEQ ID Nos. 1, 2, 3, 4, 5, 6 and 24 for detecting and predicting predisposition to immunological disorders in a subject, said markers comprising of the following characteristics:
(i) the SEQ ID No. 1 contains 1-230 contiguous nucleotides containing group of CA dinucleotides of locus M1 present 44.7 kb upstream of gene start site;
(ii) the SEQ ID No. 2 contains 1-400 contiguous nucleotides containing GT dinucleotides at locus M2;
(iii) the SEQ ID No. 3 has 1-159 contiguous nucleotides containing CA repeat polymorphism at nucleotide 229 of M3 locus;
(iv) the SEQ ID No. 4 has 1-1036 contiguous nucleotides containing A/T polymorphism at nucleotide 75 of locus S1, C/T polymorphism at nucleotide 388 of locus S2 and C/T polymorphism at nucleotide 861 of locus S3;
(v) the SEQ ID No. 5 has 1-961 contiguous nucleotides containing G/A polymorphism at nucleotide 147 of S4 locus;
(vi) the SEQ ID No. 6 has 1-1707 contiguous nucleotides containing C/T polymorphism at nucleotide 1221 of S5 locus;
(vii) the SEQ ID No. 24 has 1-817 contiguous nucleotides containing splice variants.

40. Pharmacogenetic markers as claimed in claim 39, wherein SEQ ID NO:1 is associated with micro-satellite repeat 1 as is represented in FIG. 1, SEQ ID NO:2 is associated with micro-satellite repeat 2 as is represented in FIG. 1, SEQ ID NO:3 is associated with micro-satellite repeat 3 as is represented in FIG. 1, SEQ ID NO:4 is associated with SNP 1, SNP 2 and SNP 3 as represented in FIG. 1, SEQ ID NO:5 is associated with SNP 4 as represented in FIG. 1, SEQ ID NO:6 is associated with SNP 5 locus of the INPP4A gene as represented in FIG. 1 and SEQ ID NO:24 associated with novel splice variants.

41. Pharmacogenetic markers as claimed in claim 39, wherein the subject is human.

42. A diagnostic kit comprising of pharmacogenetic markers having SEQ ID Nos. 1, 2, 3, 4, 5, 6 and 24 along with an instruction manual for detecting and predicting predisposition to immunological disorders in a subject.

43. A kit as claimed in claim 42, wherein SEQ ID NO:1 is associated with micro-satellite repeat 1 as is represented in FIG. 1, SEQ ID NO:2 is associated with micro-satellite repeat 2 as is represented in FIG. 1, SEQ ID NO:3 is associated with micro-satellite repeat 3 as is represented in FIG. 1, SEQ ID NO:4 is associated with SNP 1, SNP 2 and SNP 3 as represented in FIG. 1, SEQ ID NO:5 is associated with SNP 4 as represented in FIG. 1, SEQ ID NO:6 is associated with SNP 5 locus of the INPP4A gene as represented in FIG. 1 and SEQ ID NO:24 is associated with splice variants.

44. A kit as claimed in claim 42, wherein the subject is human.

45. A kit as claimed in claim 42, useful for predicting and detecting humans susceptible to immunological disorders selected from the group comprising of asthma, autoimmune disorders, inflammatory disorders, cancer, multiple sclerosis, fibrosis, tuberculosis, sarcoidosis, hypertension and related disorders, diabetes and related disorders, alcohol abuse, anxiety, COPD, cholecystectomy, degenerative joint disease, seizure disorder, arthritis.


SEQUENCE LISTING (26 sequences)

SEQ ID NO: 1 - DNA, 401 bp, human
  misc_feature (31)..(73): consists of CA dinucleotides (D2S2311) at nucleotides 31-73
ggggcaagtggcgatagggctttctacatacacacacacacacacacacacacacacacacacacacacacaatgatggaagtaaattagagggacatagcaaccaacctgaaagagctcccaatggccaaaactgaagccatttgaaaaacaaaatttttaaatttagtattagactatatcccaaagtataaaagtccatgagtctatgccataaatgaatgattgaataaataaattaatgagggagaaaggacacatattttttacagaaggattccagatcatatatgtagattatttgatactttccaccccaggatgtaaagccttattgatggatcttgcactcagaactggagccaaggatccagcagcctctacactgccatgggggaaca

SEQ ID NO: 2 - DNA, 240 bp, human
  misc_feature (116)..(164): consists of GT dinucleotides (D2S2187) at nucleotides 116-164
gggctggagggggtggagtagggagtgttgtgaaagtttcaagcacagaggactgaatggaagcctcacaatgcaacctaaggactttggattcttgcttttgagtacatgcactgtgtgtgtgtgtgtgtgtgtgtgtgtgtgtgtgtgtgtgtgtgtgtgtaaatggtgtagtttgttggctgtcagccttactaggaggctggtttggagcctgttgccttagctctgtggggtgat

SEQ ID NO: 3 - DNA, 159 bp, human
  misc_feature (105)..(122): consists of CA dinucleotides at nucleotides 105-122
tcacccagtaccagaccatcatcctcacataccaggagaacctgaccgacctccatcagtacagaggtgggtgcacccccatgctgtcaccacacacgcgtgcgcacacacacacacacacactctcactctcactcagtcactccctctgctttccca

SEQ ID NO: 4 - DNA, 1036 bp, human
  misc_feature: consists of 75 A/T polymorphism (rs3769712); 388 C/T polymorphism (rs3769710) and 861 C/T (rs2278208) at nucleotide 229
tgcagcagcccttagtttgtttagagaacttgattcaggaaaaaggaaccatctttggtaaaatgagagggatcatgtaagggaaagggctttgagaaaaatatttagttacgtcatgtgtcagagttaactctagttgggttagagttaaatgtacaaaatagacatcattctttttaaaggagctaaaggcggctgatgattgaacagatagagcaggaaaggacttcacaggcacgaggacagggcaaggaaatcacaaaggaaatggtcagtagatgggaccagacaaaaatttagaacctgattaagtgagaccttgtaaatagaaagcctgaagttccagggtgtccccaggttccctggatgaatcagatgagcccaggccaccatcagccacccttcgggatgtcagggtaaagctgactgaggctggggtagcatcagctgtgtcatatcaggattcaagggcttcaggaagccctggctggattccattacttggagtccaggcttaggccttgggctaatataagcctcaaggtggggtgggaaggaacagaaaagggcccagggccagaacaaggtgttctgtagtcctcactctgacacgtgtccccctcaccctctgggcaggggtgagcacaggcactggcagcagctcaggaacagcattggctcctcctcagggaggccccctctgacttcaccagcacgtccacacttccctgtggtgcgtgtccgttcagacacagccctccactgtgccccatggtttccttgtctgtctcgcctgagaggagccctgtgtgcacagtgaatgctgactgactggtcagtcagctcggccctgctgcccccaggaccctgatgacccagtacggctagggctttgtatttcagcagaaaagaagcacagttgcagcaccactcattaaagcatgttgtgcatcacaggtctgcagagagtgaccgtgtaggtaacatcaccgtgattggctggcagatggaggagaagtcagaccaacggccccct

SEQ ID NO: 5 - DNA, 961 bp, human
  misc_feature (147)..(147): consists of G/A polymorphism (rs2278206) at nucleotide 147
atggcgagggctgtgaggatgtcttcccctgtgcaggcagctgcaccagcaagaaaggtaacccggacagccacgcctactggatcagaccagaagaccccttctgtgatgtcccctcctcaccatgcccctccaccatgccctccactgcatgccatcctcatctgaccacacgtgcgtatgcatccatgcttcccatatgctgcctccatttcaccttgtgctgcttccatttgtctgtgtctctgactccatgccctcctcccccatgcagactgcagggagggctgccaggtggagcgggggcgtcccagtgcatggagcagcaggcagtgagagcggagtctccggttgcctttccatgtaactggagcccatgtgtgattctgcatctctgccagtcagaatctcagggtcctcttgagatacaccaccatggctataagggttgggctcctgggcttttcaggtttgtaaagcaatctgagaacctgaaggaggccaggatgcagatgcctttctgatgtgattgtctatggcattaggaattggtagaatgaaaggggaaaggggaattgttcagggacacagctggatgagagatattggcaggtccagtggacatgtctaaaagctggttggcattggctgggcactctcttatgcagcagaggcatgttgccttgggacttggtaacacagagaatgggaccctgttcctgggcttctggtggtctgagtcaggtggtttggcaggacccttcagggtaggacactggcatgaagcagcatgcttcagaacagtctcctgtctgcagagacacagcataccaggacactgtcctgcagatggccttaaagctgggaaacagtccctgcaactgagatgtctccgccttttcctgtaagagccatagagttacaagctaggcagctgagacttgcccacagacctagcttc

SEQ ID NO: 6 - DNA, 1707 bp, human
  misc_feature (1221)..(1221): consists of C/T polymorphism (rs10201079) at nucleotide 1221
ccttggctgcatgggttctcacgttgtccaccgaatcccaggcatcccctggggataaggggttgtggggcagcagtgccagccactcaggtactgagtccttccatccccagtttgagcagagctgctctccttttcaggctcttatttctttttgtttgtgtgtatgaataaagggtccctggctgaatagctggaatatctttagtcaggtctaagcctatatctcatggatggggagacccagcagtgcaccacaagtggctggtgtccctctgggatggaagcctggcactgcagtgccctcttgggcaaggatgcatcactggccatccctgcctgccttccagaacctcacgttagctctggatgcagcatctatgaatttaaagctttttgaaacctatagttccagcttccgtcatgtgtttgggtcatgaactctacagcccacagaatttagtgcacataccaagttcactcttgctttgtcagttacaactttacaaatttgattaatttactctccattagcttagggtctgcttatctgcttagggcctgttatttttttttctttctttttgttggtctgttctcagaagaagcctattctccttctgattgtcttgcctgaacattgggtcttttgttttgcctttccctagacgaggttcttagcactgtaccatgttcgtgtctagtgatgatcctggtagtgactggcatgctctttggctttgctggcctgacaagcaggtgttgtgccatcttcaggagttgatgagacactgatatttttctggcatacaacagatacccggagcccaccatcatatcacttggatgatgtttcattaaataccagccttggtgtcatagtgcagtatctgatacggtgcatagtcctttatctgaagcccctgtggccagatgctttggaatgcacaggttttcagattttagaaagtactgtggtacacagttggtgtttacttatgtaagtaatatggtgcccatcgcatatgtgtaccatggtatacatacaggatttaacatccccgggggagcctgagacagcactctgcaaccaagctcatattattgccacactgaaaagtatgaatatttacataaaatgggataagtaaagattgcaagtagtcttatatcagctcaggtctggttgtgctgccaaatgagattgccacagatttacatgaaaaaattaccttgtttcagagcttgatggatttggtgactgtgtgcctaggattgtatctagaggtgcagggaaaatgccctgaaatctccccatggggagaggaggtgcactcaggtggccttttcccagtccagaggctgccctgccccctactctgtgagccattcagttgtgaggagggaagtgtgctggcagtaactctgctaccgggaaagtatggcgggaggaggcctgtggggctggtggtcaggaggtggccaaagggcagtgcaacttctgagctgctcccgtctgttcaggtgaaagaggagagtagagatagtaggtctgttccctaccttccagggagacctagagacctggcccaggactgtgggcatcctgagagtggctctggcttccccagtgggtcatatttgccgccctctttccctgagggatcagcagtgaagggctgaacgtgataagagggcccaggcatg

SEQ ID NO: 7 - DNA, 18 bp, human
  misc_feature: Forward primer for SEQ ID NO1; synthetic oligonucleotide
ggggcaagtggcgatagg

SEQ ID NO: 8 - DNA, 20 bp, human
  misc_feature: Reverse primer for SEQ ID NO1; synthetic oligonucleotide
tgttcccccatggcagtgta

SEQ ID NO: 9 - DNA, 21 bp, human
  misc_feature: Forward primer for SEQ ID NO2; synthetic oligonucleotide
gggctggagggggtggagtag

SEQ ID NO: 10 - DNA, 25 bp, human
  misc_feature: Reverse primer for SEQ ID NO2; synthetic oligonucleotide
atcaccccacagagctaaggcaaca

SEQ ID NO: 11 - DNA, 23 bp, human
  misc_feature: Forward primer for SEQ ID NO3; synthetic oligonucleotide
tcacccagtaccagaccatcatc

SEQ ID NO: 12 - DNA, 22 bp, human
  misc_feature: Reverse primer for SEQ ID NO3; synthetic oligonucleotide
tgggaaagcagagggagtgact

SEQ ID NO: 13 - DNA, 24 bp, human
  misc_feature: Forward primer for SEQ ID NO4; synthetic oligonucleotide
tgcagcagcccttagtttgtttag

SEQ ID NO: 14 - DNA, 18 bp, human
  misc_feature: Reverse primer for SEQ ID NO4; synthetic oligonucleotide
agggggccgttggtctga

SEQ ID NO: 15 - DNA, 23 bp, human
  misc_feature: Forward primer for SEQ ID NO5; synthetic oligonucleotide
atggcgagggctgtgaggatgtc

SEQ ID NO: 16 - DNA, 24 bp, human
  misc_feature: Reverse primer for SEQ ID NO5; synthetic oligonucleotide
gaagctaggtctgtgggcaagtct

SEQ ID NO: 17 - DNA, 21 bp, human
  misc_feature: Forward primer for SEQ ID NO6; synthetic oligonucleotide
ccttggctgcatgggttctca

SEQ ID NO: 18 - DNA, 21 bp, human
  misc_feature: Reverse primer for SEQ ID NO6; synthetic oligonucleotide
catgcctgggccctcttatca

SEQ ID NO: 19 - DNA, 19 bp, human
  misc_feature: Snapshot primer for SNP 75 A/T (rs3769712) in SEQ ID NO4; synthetic oligonucleotide; Reverse strand
caaagccctttcccttaca

SEQ ID NO: 20 - DNA, 22 bp, human
  misc_feature: Snapshot primer for 388 C/T polymorphism (rs3769710) in SEQ ID NO4; synthetic oligonucleotide; Reverse strand
atcccgaagggtggctgatggt

SEQ ID NO: 21 - DNA, 25 bp, human
  misc_feature: Snapshot primer for 861 C/T (rs2278208) in SEQ ID NO4; synthetic oligonucleotide; Reverse strand
agccgtactgggtcatcagggtcct

SEQ ID NO: 22 - DNA, 20 bp, human
  misc_feature: Snapshot primer for C/T polymorphism (rs2278206) at nucleotide 147 in SEQ ID NO5; synthetic oligonucleotide; Reverse strand
cagatgaggatggcatgcag

SEQ ID NO: 23 - DNA, 18 bp, human
  misc_feature: Snapshot primer for C/T polymorphism (rs10201079) at nucleotide 1221 in SEQ ID NO6; synthetic oligonucleotide
gattgccacagatttaca

SEQ ID NO: 24 - DNA, 816 bp, human
  misc_feature: cDNA sequence containing splice variant region; length variable from 817-598 nucleotides
gggcgccagcagcacactgccaaggttttaagtcaggaggtctccgcaaaaagctgcacaaatttgaagagaccaagaaacattttgaggagtgttgtacatcatctggctgccagtccataatctacataccccaggatgttgtcagagccaaggagatcatcgcccagatcaacaccctgaaaacccaagtgagttactacgcagagcggctgtcaagggcagccaaggacaggtctgccactggccttgagaggacactcgccatcttggcagacaagacacggcagctggtcacggtctgcgactgcaagctcctggccaactccatccatgggctgaacgctgcacggcctgactacattgcctccaaggcctctcccacttcgactgaggaggagcaggtgatgcttagaaatgaccaggacaccctcatggcccggtggacagggagaaacagccgatcttccctgcaggtggactggcacgaggaggagtgggagaaagtgtggctgaacgtggacaagagcctagagtgcatcattcagcgtgtggacaagctgctgcagaaggagcggctgcatggcgagggctgtgaggatgtcttcccctgtgcaggcagctgcaccagcaagaaaggtaacccggacagccacgcctactggatcagaccagaagaccccttctgtgatgtcccctcctcaccatgcccctccaccatgccctctactgcatgccatcctcatctgaccacacattgcagtccccctcctgaagagtccagcccaggtgaatggagtgaggccctttacccgc

SEQ ID NO: 25 - DNA, 19 bp, human
  misc_feature: Forward primer for cDNA sequence containing splice variant region; synthetic oligonucleotide
ggggcgccagcagcacact

SEQ ID NO: 26 - DNA, 24 bp, human
  misc_feature: Reverse primer for cDNA sequence containing splice variant region; synthetic oligonucleotide
gcgggtaaagggcctcactccatt